(19) United States
(12) Patent Application Publication
(10) Pub. No.: US 2016/0235296 A1
(43) Pub. Date: Aug. 18, 2016

(54) DETECTION OF NUTRIENT DEFICIENCIES INFLUENCING OCULAR HEALTH

(71) Applicant: Barbara L. DUNNING, Toronto (CA)

(72) Inventor: Barbara L. DUNNING, Toronto (CA)

(21) Appl. No.: 15/025,811

(22) PCT Filed: Sep. 30, 2014
(86) PCT No.: PCT/CA2014/050938

§ 371 Date: Mar. 29, 2016

Related U.S. Application Data
(60) Provisional application No. 61/884,623, filed on Sep. 30, 2013.

(51) Int. Cl. A61B 3/12; A61B 5/00; A61B 5/103; A61B 3/14; A61B 3/00

(52) U.S. Cl. CPC A61B 3/12; A61B 3/14; A61B 3/0025; A61B 5/1032; A61B 5/4869

(57) ABSTRACT

The present invention provides methods for identifying and treating subjects having nutrient deficiencies. A method for scoring nutritional deficiency in a subject's eye is also provided.

### CROSS REFERENCE TO PRIOR APPLICATIONS

This application claims priority under the Paris Convention to U.S. Provisional Patent Application 61/884,623 filed Sep. 30, 2013 which is incorporated herein by reference.

### FIELD OF THE INVENTION

The field of invention relates to methods and compositions useful for detecting, diagnosing, preventing and treating ocular diseases and corresponding nutrient deficiencies and microvascular non-perfusion.

### BACKGROUND OF THE INVENTION

Ocular health and disease is a global concern, particularly given the aging population in many countries. Ocular health is thought to decrease naturally with age, and can be compromised by oxidative stress, illness and visual stresses, such as prolonged exposure to visual display monitors (U.S. Patent Application Publication No 2012/0258168).

Nutrition is one feature of ocular health that has been studied in age-related ocular diseases, such as age-related macular degeneration (AMD). Macular degeneration is a chronic eye disease that causes vision loss in the central field of vision. Dry macular degeneration is marked by deterioration of the deep layers of the retina. Wet macular degeneration is characterized by blood vessels that grow under the retina, leaking blood and fluid. The pathology of AMD is believed to be caused, at least in part, by oxidative damage (Beatty et al., Surv. Opthamol. 2000, 45:115-134; Cai et al., Prog. Retin. Eye Res. 2000, 29:263-271). The healthy eye contains antioxidant molecules, including enzymes, vitamins C and E, omega-3 fatty acid docosahexanic acid (DHA) and macular pigments lutein and zeaxanthin. Deficiency of antioxidants in the aging eye is believed to be a risk factor for development of AMD (Ocular Nutrition: It's Role in Maintaining Eye Health, Module 1: Nutrition and Health of the Aging Eye,2011, 6 pages). It follows that nutrient supplements, including antioxidants such as, zinc, vitamin C, vitamin E, beta carotene, lutein, zeaxanthin and omega-3 fatty acids, are sometimes recommended to prevent AMD progression and improve vision.

Lutein and zeaxanthin are xanthophyll carotenoid pigments found in the retina. Subjects having AMD are known to have decreased amounts of lutein and zeaxanthin in their retina. Some studies suggest that visual acuity, contrast sensitivity, and the amount of retinal pigment in the human eye can be improved as a result of lutein and zeaxanthin supplementation or a combination of these xanthophylls with other antioxidants (Stiles et al. (2004) Optometry, 75:216-230). Other studies suggest that macular pigment optical density (MPOD), a measure of the amounts of lutein and zeaxanthin in the macula of the living human eye, is a marker of the health of the human eye (U.S. Patent Application Publication No. 2012/0070422).

Nutritional supplements including lutein and zeaxanthin have also been suggested to promote ocular health and treat “ocular diseases” (see, for example, U.S. Patent Application Publication Nos. 2010/0068298 and 2012/0258168). However, the range of “ocular diseases” appears to be limited to early stages of AMD and related ocular disorders thought to be associated with oxidative stress (U.S. Patent Application Publication No. 2010/0068298). The favored dosage of lutein and zeaxanthin has been 10 and 2 mg, respectively, often provided to subjects in combination with omega-3 fatty acids and one or more antioxidant nutrients, such as Vitamin C, Vitamin E and zinc, which were included in the AREDS 2, wherein the composition was tested on patients having moderate to severe AMD (e.g., The AREDS2 Research Group, JAMA 309:2005-2015, 2013). However, these nutrients are not known to reduce the risk of progression to advanced AMD or to treat moderate to severe AMD.

Methods for identifying nutritional deficiencies by way of an eye exam are desirable. Nutritional treatment of nutritional deficiencies identified by way of an eye exam is desirable.

### SUMMARY OF THE INVENTION

In a first aspect, the present invention provides a method for identifying a subject having a nutritional deficiency. The method comprises obtaining an image of the subject's ocular posterior pole; comparing the image to at least one reference ocular posterior pole image; and identifying a nutritional deficiency in the subject based on the comparison to the reference image.

In some embodiments color retinal photographs are converted to grayscale images of the choroid, the retina and the RNFL (retinal nerve fiber layer). The RNFL grayscale images of a subject are compared to reference RNFL grayscale images.

In some embodiments, if the reference image is a healthy posterior pole, then an obtained image that comprises at least one region of increased contrast relative to the reference image is indicative of the nutrient deficiency. In some embodiments, if the reference image is a nutrient deficient posterior pole, then an obtained image that comprises at least one region of equal or increased contrast relative to the reference image is indicative of the nutrient deficiency. In some embodiments, the nutrient deficiency comprises a deficiency of one or more of lutein and zeaxanthin.

In some embodiments, the method further comprises monitoring a subject for nutritional deficiency. In some embodiments of the monitoring method, a subsequent image is obtained from the subject's posterior pole and compared with an image obtained from the subject at an earlier point in time. In some embodiments, if the subsequent image depicts increased contrast relative to the earlier image, then the subsequent image is indicative of lower nutrient levels in the subject's posterior pole. In some embodiments, if the subsequent image depicts decreased contrast relative to the earlier image, then the subsequent image is indicative of higher nutrient levels in the subject's posterior pole.

In some embodiments, the method further comprises assigning a score to the obtained image, wherein the score is based on a comparison between the obtained image and a reference database comprising posterior pole images obtained from a range of nutrient sufficient (healthy) and nutrient deficient subjects, wherein scores at opposite ends of the range are indicative of high nutrient levels (very healthy) and severely nutrient deficient subjects, respectively. In some embodiments, the assigned score is indicative of lutein and zeaxanthin levels in the subject's posterior pole.

### BRIEF DESCRIPTION OF THE DRAWINGS

The features of the invention will become more apparent in the following detailed description in which reference is made to the appended drawings wherein:

FIGS. 1A-H are ocular images obtained from glaucoma patients.

FIGS. 2A-H are ocular images obtained from geographic atrophy (GA) patients.

FIGS. 3A-L are ocular images obtained from diabetic retinal fibrosis patients.

FIGS. 4A-K are ocular images obtained from a patient having retinal RNFL fibrosis.

FIGS. 5A-Q are ocular images obtained from wet AMD patients.

FIGS. 6A-T are ocular images obtained from VRT patients.

FIGS. 7A-H are ocular images obtained from CRVO/BRVO patients.

FIGS. 8A-F are ocular images obtained from a pre-retinal detachment patient.

FIGS. 9A-D are ocular images depicting reversal of arterial sclerosis of the central retinal arteries.

FIGS. 10A-D are ocular images obtained from a subject having presumed arterial sclerosis of the choroidal arteries.

FIGS. 11A-D are ocular images obtained from a subject having epiretinal membrane.

FIGS. 12A-D are ocular images obtained from a subject having early low-tension glaucoma.

### DETAILED DESCRIPTION OF THE INVENTION

The definitions of certain terms as used in this specification are provided below. Unless defined otherwise, all technical and scientific terms used herein generally have the same meaning as commonly understood by one of ordinary skill in the art to which this invention belongs.

As used herein, the term “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent to a subject includes any route of introducing or delivering to a subject a compound to perform its intended function.

Administration can be carried out by any suitable route, including orally, sublingually, intraocularly, intranasally, intravenously or topically. Administration includes self-administration and the administration by another.

The terms “comprise”, “comprises”, “comprised” or “comprising” may be used in the present description. As used herein (including the specification and/or the claims), these terms are to be interpreted as specifying the presence of the stated features, integers, steps or components, but not as precluding the presence of one or more other feature, integer, step, component or a group thereof as would be apparent to persons having ordinary skill in the relevant art.

As used herein the term “end organ” or “EO” refers to a tissue that is supplied by small-diameter arteries and capillaries of that organ, wherein the tissue is the terminal delivery point of a given artery or capillary.

As used herein, the term “lesion” refers to a localized change in an organ or tissue of the body. “Retinal lesions”, referred to herein, can be characterized by at least one of puckering, fibrosis or gliosis, lamellar splitting, retinal dragging and/or swelling, bulging of retinal tissues, retinal holes, edema, swelling, exudates, deposits, hemorrhaging and atrophy.

As used herein the term “microvascular non-perfusion” or “MVNP” refers to incomplete filling and/or emptying of blood in small-diameter arteries and capillaries.

As used herein, the term “nutraceutical” refers to specific chemical compounds found in foods that can prevent disease or ameliorate an undesirable condition.

As used herein, the term “nutritional deficiency disorder” refers to an impairment of normal physiological function of any tissue of the eye, wherein the cause of impairment is the lack of one or more nutrients.

As used herein, the term “Oculus Dexter” or “OD” refers to the right eye of a subject.

As used herein, the term “Oculus Sinister” or “OS” refers to the left eye of a subject.

As used herein, the term “omega 3 fatty acids” refers to fats commonly found in marine and plant oils, such as fish oils, algal oil, squid oil, echium oil and flaxseed oil.

Examples of omega 3 fatty acids useful in the present invention include, but are not limited to, eicosapentaenoic acid (EPA) and docosahexaenoic acid (DHA).

As used herein the term “probiotic” refers to one or more strain of live microorganisms that may confer a health benefit on their host. Probiotics can be consumed as part of fermented foods or as dietary supplements. Examples of probiotic organisms include some members of the Order Lactobacillales, such as Lactobacillus spp. And members of the genus Bifidobacterium.

As used herein, the term “retinal disorder” or “disorder of the retina” refers to any impairment of normal physiological function of the retina.

As used herein, the term “therapeutically effective amount” refers to a quantity sufficient to achieve a desired therapeutic and/or prophylactic effect, e.g., an amount which results in the prevention of, or a decrease in, the symptoms associated with a retinal disorder. For example, a “therapeutically effective amount” of the composition of the present invention refers to levels of the composition that, when administered to the subject on a daily basis, ameliorate, in part or in full, at least one symptom of the disorder, for example, the size of a retinal lesion.

As used herein, the terms “treating” or “treatment” or “alleviation” refers to both therapeutic treatment and prophylactic or preventative measures, wherein the object is to reverse, prevent or slow down (lessen) the targeted pathologic condition or disorder. A subject is successfully “treated” for an ocular disorder if, after receiving an effective therapeutic amount of the composition according to the methods described herein, the subject shows measurable reduction in at least one symptom or sign of an ocular disorder. It is also to be appreciated that the various modes of treatment or prevention of medical conditions as described are intended to mean “substantial”, which includes total but also less than total treatment or prevention, and wherein some biologically or medically relevant result is achieved.

As used herein, the terms “units of diameter” and units made in reference to lesion size or diameter refer to units of diameter of the posterior pole of an eye, which measures ten units in diameter between the vascular arcades. A lesion can be larger than 10 units.

As used herein, the terms “vitreomacular traction,” (VMT) and “vitreoretinal traction disorder” (VRTD) refer to conditions wherein the membrane of the vitreous gel of the human eye adheres to the retina, causing pulling, or “traction”, forces on the retina that can cause ocular damage.

Abbreviations and Acronyms

In addition to the above noted definitions the following abbreviations and acronyms may appear in the application:
CRA—central retinal artery
choroid-choriocapillaris
ONH—optic nerve head
RPE—retinal pigment epithelium disruption
GA—Geographic Atrophy (of all retinal layers)
OU—both eyes
RTC—return check-up
IOL—intraocular lens
CWS—cotton wool spot
e/d/360/240—dose comprising 360 mg of EPA and 240 mg of DHA.
e/d/180/120—dose comprising 180 mg of EPA and 120 mg of DHA
DD—disc diameter
ERM—epiretinal membrane
CSR—central serous retinopathy
pre-ERM—pre-epiretinal membrane
pre-CRS—pre-central serous retinopathy
rpe—retinal pigment epithelium
VA LP—visual acuity of light perception
CCA—common carotid artery
ICA—internal carotid artery
CME—central macular edema
CRVO—central retinal vein occlusion
BRVO—branch retinal vein occlusion

The present invention is based on the inventor's observations of subjects having various ocular diseases. Upon treatment with nutrient supplements, including at least about 20 mg lutein and at least about 5 mg zeaxanthin, the inventor observed alleviation of various ocular disorders in the subjects. Further, the inventor began documenting characteristics of the retina and the retinal nerve fiber layer (RNFL) in subject's ocular posterior poles before and after treatment. An emerging trend in these characteristics led the inventor to develop a method of identifying nutrition deficiency disorders (NDDs) in subjects by way of an eye examination.

In one aspect of the present invention, a method for identifying a subject having a NDD is provided. The method involves a non-invasive eye examination using a camera, such as a retinal camera, and software capable of imaging the posterior pole of the eye. The skilled artisan is aware of various cameras and software for imaging the posterior pole of the eye, including, but not limited to EyeScape Digital Imaging Software. The image of the subject's posterior pole is then compared to a predetermined reference standard. In some embodiments, the predetermined reference standard is a reference database. The database comprises various photos of posterior poles in subjects having a range of ocular nutrient deficiencies and states of health. Based on the comparison to the reference standard, the image generated is assigned a score. The assigned score may be called a Nutritional Deficiency Score (NDS). In some embodiments, the score is an integer from 1 to 5, wherein 1 indicates a healthy eye having good nutritional health and 5 indicates a subject having severe nutrient deficiency. A subject having severe nutrient deficiency, e.g., a score of 4 to 5 is typically at high risk for eye disease or is already suffering from eye disease. A subject having a score of three is at lower risk for eye disease but might be prescribed a nutrient composition to prevent development of eye disease. A score of 1 or 2 indicates a healthy eye having good nutritional health. In some embodiments, the scale used to score nutrient deficiency is generally linear rather than exponential or logarithmic, for example. In some embodiments color retinal photographs from the subject are converted to grayscale images of the choroid, the retina and the RNFL (retinal nerve fiber layer). The RNFL grayscale images of a subject are then compared to reference RNFL grayscale images.

It is contemplated herein that a reverse numbering scale could be used, in which increased scores over time would indicated improved ocular health and nutrient levels. Further, it is contemplated that non-linear scoring systems could be used to achieve a similar characterization of ocular health and/or nutrient deficiency.

In some embodiments of the present invention, the nutrient deficiency is primarily a lutein and zeaxanthin deficiency. In some embodiments a score indicating combined lutein and zeaxanthin sufficiency or deficiency is provided. In some embodiments a score of lutein deficiency is provided. In some embodiments a score of zeaxanthin deficiency is provided. Lutein and zeaxanthin are distributed in different zones of the retina. Lutein deficiencies are identified within the vascular arcades with the exception of the macula itself. Zeaxanthin deficiencies are identified beyond the vascular arcades and in the macula itself. Zeaxanthin collects in the center of the macula while lutein is distributed throughout the rest of the retina.

A nutritional deficiency score of zeaxanthin alone would reflect the relative level of pigmentation in the zeaxanthin zone. A nutritional deficiency score of lutein alone would reflect the relative level of pigmentation in the lutein zone. A combined lutein and zeaxanthin nutritional deficiency score would reflect the relative level of pigmentation of the entire retina of a subject.

In another aspect of the present invention, a method for treating a nutrient deficiency in a subject is provided. The method involves generating an image of a subject's posterior pole, comparing the generated image to a reference and assigning a score to the generated image based on the comparison. In some embodiments, if the subject's posterior pole image has a score indicative of a nutrient deficiency then the subject is treated with a nutraceutical composition for a period of at least two weeks. In some embodiments, the nutrient deficient subject will be administered daily with at least about 20 mg lutein and at least about 5 mg zeaxanthin. In preferred embodiments, the nutrient deficient subject will be administered daily with at least about 20 mg lutein, at least about 5 mg zeaxanthin, at least about 180 mg omega-3 fatty acids (e.g., EPA) and at least about 120 mg DHA. In some embodiments, the nutrient deficient subject will be administered daily following supper comprising protein with “LZO3P” therapy comprising 20 mg lutein, 5 mg zeaxanthin, 180 mg EPA omega-3 fatty acids and 120 mg DHA.

In some embodiments, the treated subject will have a further eye examination following treatment, wherein a second NDS is generated. Preferably, the second NDS is compared to both the first NDS and the corresponding reference standard(s). In this way, the scores before and following treatment with the nutraceutical composition can be compared. Assuming the 1 to 5 scoring scale discussed above, a decreased score following treatment may accompany an improvement in nutrient sufficiency while an increased score following treatment would indicate a worsening nutrient deficiency. Assuming the 1 to 5 scoring scale discussed above, a decreased score following treatment would further indicate an improvement in ocular health while an increased score following treatment would indicate a worsening ocular health.

In some embodiments of the present invention, the composition is administered to the subject orally. The compositions of the invention can be formulated with suitable carriers such as starch, sucrose or lactose in tablets, capsules, solutions, powders, syrups and emulsions, or oils. Suitable optional carriers include but are not limited to, for example, fatty acids, esters and salts thereof, that can be derived from any source, such as for example, natural or synthetic oils, fats, waxes or combinations thereof. In preferred embodiments of the present invention, the source of the fatty acids is DHA and EPA, which may be provided in combination with the composition or separately.

In some embodiments of the present invention, the composition is administered daily for two to 24 months to the subject. In preferred embodiments, the composition is administered daily to the subject for 3 months. In particularly preferred embodiments of the present invention, the composition is administered to the subject daily until ocular disease(s) are ameliorated completely and/or the subject has an NDS of 1, 2 or 3.

In another aspect of the present invention, a method for monitoring a nutrient deficiency in a subject is provided. In some embodiments, the method of monitoring involves repeatedly obtaining nutritional deficiency scores from a subject over time, using the method disclosed above. Repeated measurements can allow comparison to previous states of eye health and/or disease and to a reference. Further, repeated measurements can allow evaluation of various treatments for eye health and disease.

It is contemplated herein that the present methods of treating and monitoring nutrient deficiencies, can be useful for improving various ocular disorders, including, but not limited to, one or more of AMD, (dry and wet), vitreoretinal traction disorder (VRTD), central macular edema, CME, diabetic macular edema, DME, diabetic retinopathy, retinal hemorrhages, sub-choroidal hemorrhages, cotton wool spots, retinal exudates, low-tension glaucoma, cataract, open angle glaucoma, miosis, iris edema, angle closure glaucoma, corneal dystrophy, corneal guttata, pterygia secondary to xerophthalmia, meibomian gland dysfunction, recurrent eyelid styes, recurrent chalazia, severe dry eye, and eyelid ectropion. Diagnosis can be made using methods known to those of skill in the art, at least for example, slit lamp biomicroscopy, retinal photography, visual acuity, Amsler grid intraocular pressure, central corneal thickness or fundus examination.

Without being bound to any one theory, it is contemplated herein that the nutrient deficiency that is characterized by a darkening of at least a portion of the RNFL of a subject's eye is associated with microvascular non-perfusion. It is contemplated that microvascular nonperfusion limits blood flow and, consequently limits nutrient delivery to and waste removal from end organ locations, such as the eye. It is contemplated that microvascular nonperfusion-associated darkening of a subject's posterior pole might be otherwise asymptomatic. It is contemplated that reversal of microvascular nonperfusion, for example by way of nutrient therapy, might prevent or reverse various eye diseases.

It is contemplated herein that unilateral eye disease may occur secondarily to asymptomatic carotid stenosis and that one or more of the internal, external and common carotid arteries may be involved. It is contemplated herein that internal carotid stenosis can result in one or more of the following: AMD, (dry and wet), VRTD, central macular edema, diabetic macular edema, diabetic retinopathy, retinal hemorrhages, sub-choroidal hemorrhages, cotton wool spots, retinal exudates, low-tension glaucoma, mini-BRVO, cataract, open angle glaucoma, miosis, iris edema, angle closure glaucoma, corneal dystrophy, corneal guttata, pterygia secondary to xerophthalmia, meibomian gland dysfunction, recurrent eyelid styes, recurrent chalazia, severe dry eye, and eyelid ectropion. It is contemplated that external carotid stenosis can result in one or more of the following: reduced lacrimal and salivary gland secretions, meibomian gland dysfunction, recurrent eyelid styes, recurrent chalazia, severe dry eye and eyelid ectropion.

### EXAMPLES

The present invention is further illustrated by the following examples, which should not be construed as limiting in any way.

### Example 1

### Materials and Methods

Imaging of subject posterior poles: retinal photographs were taken using a Canon CR-1 Digital Retinal Camera and analyzed using EyeScape Digital Imaging Software, version 7.5.5.

The settings used for taking retinal photographs were such that optimal contrast and brightness for each subject was achieved. Most retinal photographs were taken with a dilated pupil. Some subjects have naturally very large pupils. In these cases, pupil dilation may not have been required to obtain sufficient images. The Canon CR-1 has a setting for small pupils that was used as required.

Determination of retinal lesion size: Retinal photographs were examined for signs of ocular disease. VRTD lesion size was measured directly from the computer screen display of a subject's retinal photograph. One “unit” was equivalent to one tenth of the diameter of the posterior pole, within the vascular arcades.

Recording of other health characteristics: non-ocular health conditions were, in some cases, reported by subjects. These conditions were recorded.

Nutrient supplements and administration protocol:

### Example 2

### Retinal Photograph Analysis Indicated a Positive Correlation Between VRTDs and High NDSs and Lutein and Zeaxanthin Treatment Resulted in Decreased NDS and Lesion Size

Subject Description: Data were collected from 40 subjects having asymptomatic or symptomatic VRTD over a period of 4.5 years.

Treatment: Subjects were treated daily with lutein and zeaxanthin as described in table 1. Nutritional deficiency scores (NDSs) and lesion sizes were measured at various time points. The nutritional deficiency scoring rubric involves a scoring system of 1 to 5, wherein 1 indicates a healthy eye having sufficient lutein and zeaxanthin and 5 indicates a severely nutrient deficient eye, wherein lutein and zeaxanthin levels are low relative to a healthy eye and correlate with ocular disease.

TABLE 1
Raw data collected from subjects during preliminary lutein zeaxanthin trial. Combined Lutein (L) and
Zeaxanthin (Z) NDSs are provided for each eye (OD and OS). Plh indicates partial lamellar hole. Pucker
(macular) is described based on a qualitative score of mild to severe, wherein a score of 1of 3 is a
mild pucker, a score of 2 of 3 is a moderate pucker and a score of 3 of 3 is a severe pucker.

| Case | | | Exam | lutein | Zeaxanthin | OD | OD | OS | OS | |
|---|---|---|---|---|---|---|---|---|---|---|
| # | M/F | DOB | Date | (mg) | (mg) | NDS | lesion | NDS | lesion | Comments |
| 730 | M | 1939 | July 2008 | 0 | 0 | | 1.5 | | 6 | NAD = normal |
| 1 | | | October 2009 | 10 | 2 | 2 | 0.5 | 2 | 5 | Self-treated |
| | | | November 2012 | 0 | 0 | 3 | 4.5 | 4 | 8 | non-compliance |
| | | | November 2011 | 10 | 2 | 2 | 2 | 2 | 4 | complied |
| | | | May 2012 | 20 | 5 | n/a | 0.5 | n/a | 0 | increased dose |
| | | | October 2012 | | | 2 | 0.5 | 2 | 0 | |
| 402 | F | 1959 | April 2009 | 0 | 0 | 2 | 0 | 2 | 8.5 | |
| 2 | | | November 2010 | 20 | 0 | 3 | 0 | 3 | 3 | |
| | | | March 2012 | 20 | 5 | 4 | 0 | 4 | 0 | |
| | | | September 2012 | 0 | 0 | n/a | 1 | n/a | 0.5 | regression next 5.7/1.4 |
| 378 | M | 1959 | May 2010 | 0 | 0 | 4 | 9 | 4 | 0 | |
| 3 | | | June 2011 | 0 | 0 | 4 | 11.5 | 4 | 0 | |
| | | | December 2011 | 2.5 | 0.625 | 3 | 10 | 4 | 0 | |
| | | | September 2012 | 0 | 0 | 4 | 14 | 4 | 0 | |
| | | | February 2013 | 20 | 5 | 4 | 14 | 4 | 0 | cont. 20/5 |
| 1562 | F | 1951 | August 2010 | 0 | 0 | 3 | 5 | 3 | 0 | |
| 4 | | | July 2012 | 0 | 0 | 3 | 5 | 3 | 0 | |
| | | | March 2013 | 20 | 5 | 4 | 4 | 3 | 0 | multi strain probiotic next |
| 1556 | F | 1941 | July 2010 | 0 | 0 | 2 | 4.5 | 2 | 11.5 | |
| 5 | | | January 2011 | 5 | 1 | 3 | 4 | 2 | 12 | |
| | | | August 2011 | 20 | 0.8 | 4 | 7 | 2 | 12 | |
| | | | August 2012 | 20 | 0.8 | 3 | 6.5 | 3 | 11.5 | Next 20/5-no rtc yet |
| 1895 | M | 1950 | February 2011 | 0 | 0 | 4 | 7 | 4 | 7 | |
| 6 | | | August 2011 | 20 | 5 | 3 | 6.5 | 3 | 7 | |
| | | | July 2012 | 20 | 5 | 3 | 4 | 3 | 7 | slow or non- responder-no |
| | | | | | | | | | | rtc yet - probiotics next? |
| 1997 | F | 1958 | May 2011 | 0 | 0 | 2 | 5 | 2 | 0 | |
| 7 | | | November 2011 | 0 | 0 | 4 | 8 | 4 | 0 | |
| | | | May 2012 | 0 | 0 | 3 | 8.5 | 3 | 0 | Next 20/5- no rtc yet |
| 294 | M | 1928 | January 2011 | 10 | 0.5 | 4 | 11.5 | 4 | 0.5 | AMD OD/IOL OU |
| 9 | | | July 2011 | 10 | 0.5 | 3 | 7.5 | 3 | 0.5 | |
| | | | January 2012 | 30 | 1.3 | 3 | 8.5 | 3 | 0.5 | |
| | | | August 2012 | 25 | 1.05 | 5 | 7.5 | 3 | 0.5 | |
| | | | February 2013 | 30 | 5.5 | 3 | 7 | 3 | 8 | right stenosis reversed |
| 1132 | F | 1949 | January 2010 | 0 | 0 | 3 | 5.5 | 3 | 1.5 | |
| 10 | | | July 2010 | 0 | 0 | 2 | 8.5 | 3 | 3.5 | |
| | | | January 2011 | 5 | 1 | 4 | 8.5 | 4 | 5.5 | cancelled rtc |
| 1660 | F | 1945 | October 2010 | 0 | 0 | 4 | 7.5 | 4 | 0 | |
| 11 | | | April 2011 | 0 | 0 | 4 | 7.5 | 3 | 0 | |
| | | | October 2011 | 0 | 0 | 3 | 7.5 | 3 | 0 | |
| | | | April 2012 | 20 | 5 | 4 | 8 | 4 | 0 | more meat/less fish/next |
| | | | | | | | | | | more fish less meat |
| | | | | | | | | | | advised/20/5 |
| | | | April 2013 | 20 | 0 | 4 | 7 | 2 | 0 | no Z = continued right |
| | | | | | | | | | | carotid stenosis, next 20/5 |
| 2010 | F | 1945 | May 2011 | 10 | 2 | 4 | 5.5 | 4 | 12 | AMD OU |
| 12 | | | December 2011 | 10 | 2 | 3 | 11 | 5 | 11.5 | |
| | | | July 2012 | 22.5 | 5.5 | 3 | 0 | 2 | 13.5 | |
| | | | January 2013 | 5 | 0.25 | 4 | 4 | 3 | 6 | Increase drusen OU |
| 483 | M | 1931 | April 2010 | 0 | 0 | 3 | 8.5 | 3 | 0.5 | |
| 13 | | | May 2011 | 0 | 0 | 4 | 9.5 | 4 | 0 | |
| | | | November 2011 | 0 | 0 | 4 | 9 | 5 | 0 | took 1 bottle 10/2.5 before |
| | | | | | | | | | | November 2011 |
| | | | May 2012 | 18 | 0 | 3 | 0 | 4 | 0 | |
| 1423 | M | 1946 | May 2010 | 0 | 0 | 2 | 0.5 | 2 | 0 | |
| 14 | | | May 2012 | 0 | 0 | 3 | 12 | 2 | 0 | 2 years later/refuses 20/5 |
| | | | November 2012 | 0 | 0 | 4 | 12 | 4 | 0 | Refuses 20/5 bilateral |
| | | | | | | | | | | stenosis? |
| 353 | F | 1934 | March 2009 | 0 | 0 | 2 | 8.5 | 2 | 9 | |
| 15 | | | April 2010 | 10 | 0.5 | 2 | 10 | 2 | 11 | AMD OU |
| | | | May 2011 | 7.5 | 0.37 | 3 | 9.5 | 3 | 8.5 | |
| | | | May 2012 | 5 | 1 | 3 | 9.5 | 3 | 9 | next 20/5 |
| | | | December 2013 | 60 | 15 | 4 | 9.5 | 3 | 9.5 | Mental confusion = wrong |
| | | | | | | | | | | dose = still no improvement = |
| | | | | | | | | | | start probiotics |
| | | | April 2013 | 20 | 5 | 2 | 0.5 | 3 | 8 | 20/5 & probiotics reverse |
| | | | | | | | | | | od VRT and right stenosis |
| 1688 | F | 1949 | December 2013 | 0 | 0 | 3 | 0 | 3 | 0 | |
| | | | April 2013 | 0 | 0 | 3 | 0 | 4 | 13.5 | |
| 16 | | | October 2012 | 20 | 5 | 2 | 11.5 | 3 | 14.5 | |
| | | | November 2013 | 20 | 5 | 3 | 10 | 4 | 9.5 | probiotics started |
| 1274 | M | 1941 | March 2010 | 0 | 0 | 4 | 7.5 | 4 | 0.5 | |
| 17 | | | September 2010 | 0 | 0 | 1 | 6.5 | 1 | 0.5 | cancer/started taking |
| | | | | | | | | | | many supplements/no |
| | | | | | | | | | | lutein/no zeaxanthin |
| | | | April 2011 | 0 | 0 | 2 | 0 | 3 | 0 | |
| | | | September 2011 | 0 | 0 | 2 | 0.5 | 4 | 0 | Cancelled April 2012 -left |
| | | | | | | | | | | stenosis |
| 242 | M | 1943 | February 2009 | 0 | 0 | 2 | 6.5 | 2 | 9.5 | |
| 18 | | | July 2010 | 0 | 0 | 3 | 6.5 | 3 | 10.5 | |
| | | | January 2011 | 10 | 2 | 3 | 6.5 | 3 | 9.5 | |
| | | | July 2011 | 10 | 2 | 3 | 2 | 2 | 8.5 | |
| | | | January 2012 | 10 | 2 | 4 | 0.5 | 4 | 8 | |
| | | | June 2012 | 20 | 5 | 4 | 0.5 | 4 | 8 | cont. 20/5 |
| | | | December 2013 | 20 | 5 | 4 | 0.5 | 4 | 4 | probiotics started |
| | | | March 2013 | 20 | 5 | 3 | 0 | 3 | 0.5 | |
| 895 | M | 1941 | September 2008 | 0 | 0 | | 0 | | 0 | no photos until November 2009 |
| 19 | | | September 2009 | 0 | 0 | 1 | 0.5 | 2 | 0 | OS heme |
| | | | January 2010 | 0 | 0 | 2 | 0.5 | 3 | 0.5 | diabetic |
| | | | September 2010 | 0 | 0 | 2 | 0 | 4 | 7.5 | |
| | | | October 2011 | 10 | 2 | 1 | 0 | 4 | 8.5 | |
| | | | April 2012 | 20 | 5 | 1 | 0 | 2 | 0.5 | used only 2 months- |
| | | | | | | | | | | reversed left VRT, |
| | | | | | | | | | | diabetes and left stenosis |
| | | | October 2012 | 20 | 5 | 1 | 0 | 1 | 0.5 | gall bladder surgery |
| | | | April 2013 | 20 | 5 | 1 | 0 | 1 | 0 | |
| 615 | F | 1944 | June 2009 | 10 | 0.5 | 1 | 0.5 | 0 | 0 | OS AMD |
| 20 | | | December 2009 | 10 | 0.5 | 4 | 0 | 4 | 0 | |
| | | | June 2010 | 5 | 0.25 | 3 | 10.5 | 3 | 0 | |
| | | | December 2010 | 5 | 0.25 | 4 | 8 | 4 | 0 | |
| | | | July 2012 | 5 | 0.25 | 4 | 0.5 | 4 | 0 | |
| | | | April 2013 | 20 | 5 | 2 | 0 | 2 | 0 | |
| 619 | F | 1948 | June 2009 | 0 | 0 | 3 | 0 | 3 | 11 | |
| 21 | | | June 2011 | 20 | 0.8 | 3 | 0 | 3 | 8.5 | |
| | | | February 2012 | 20 | 0.8 | 1 | 0.5 | 2 | 7.5 | next 20/5 |
| | | | September 2012 | 20 | 0.8 | 2 | 0.5 | 3 | 7.5 | |
| | | | April 2013 | 20 | 5 | 4 | 0 | 3 | 8 | needs probiotics |
| 181 | F | 1926 | January 2009 | 0 | 0 | 1 | 0 | 1 | 0 | IOL OU, OS 1 CWS |
| 22 | | | September 2009 | 2.5 | 0.125 | 1 | 4 | 1 | 0 | |
| | | | February 2010 | 0 | 0 | 2 | 4 | 2 | 0 | CME OU, using unknown |
| | | | | | | | | | | product |
| | | | April 2011 | 5 | 1 | 1 | 4.5 | 1 | 0 | next 20/5 |
| 127 | F | 1946 | March 2010 | 0 | 0 | 3 | 0.5 | 4 | 0 | OS drusen |
| 23 | | | October 2010 | 0 | 0 | 3 | 8 | 4 | 0 | |
| | | | April 2011 | 0 | 0 | 3 | 7 | 3 | 0 | e/d/360/240 |
| | | | November 2011 | 20 | 5 | 3 | 6.5 | 3 | 0 | e/d/180/120 |
| | | | April 2012 | 20 | 5 | 3 | 0.5 | 3 | 0 | drusen gone/e/d/180/120 |
| 2038 | F | 1954 | June 2011 | 0 | 0 | 3 | 11.5 | 2 | 0 | |
| 24 | | | December 2011 | 20 | 5 | 3 | 8.5 | 2 | 0 | e/d/360/240 |
| | | | July 2012 | 20 | 5 | 3 | 6 | 3 | 0 | e/d360/240 slow |
| | | | | | | | | | | responder/compliance? |
| | | | January 2013 | 20 | 5 | 3 | 5 | 4 | 0 | Left stenosis, needs |
| | | | | | | | | | | probiotics |
| 725 | M | 1949 | July 2009 | 0 | 0 | n/a? | | 3 | 0 | e/d/180/120 |
| 25 | | | March 2012 | 0 | 0 | 5 | 12 | 3 | 0 | e/d/360/240 right stenosis |
| | | | | | | | | | | caused cataract, then |
| | | | | | | | | | | severe ERM OD only |
| 1343 | M | 1929 | April 2010 | 0 | 0 | 2 | 4.5 | 2 | 2 | OS macular drusen ⅓ |
| | | | | | | | | | | DD size |
| 26 | | | April 2011 | 10 | 0.5 | 3 | 4 | 2 | 0 | OS macular drusen ⅓ |
| | | | | | | | | | | DD size, now ½ resolved |
| | | | | | | | | | | in retina layer |
| | | | October 2011 | 10 | 2.5 | 2 | 0 | 1 | 0 | OS macular drusen ⅓ |
| | | | | | | | | | | DD size, now 90% |
| | | | | | | | | | | resolved in retina layer |
| | | | April 2013 | 10 | 2.5 | 3 | 0 | 1 | 0 | right stenosis reversed |
| | | | January 2013 | 10 | 0 | 3 | 0 | 3 | 0 | OS macular drusen, now |
| | | | | | | | | | | getting worse with |
| | | | | | | | | | | stenosis of blood supply OU |
| 74 | F | 1951 | December 2008 | 0 | 0 | 3 | 0.5 | 3 | 0.5 | |
| 27 | | | January 2010 | 5 | 0.25 | 3 | 0.5 | 3 | 0.5 | e/d180/120 |
| | | | July 2010 | 5 | 0.25 | 3 | 2 | 3 | 2 | e/d/180/120 |
| | | | March 2011 | 5 | 0.25 | 4 | 7.5 | 4 | 6.5 | e/d/180/120 |
| | | | December 2011 | 5 | 0.25 | 3 | 5 | 3 | 8.5 | no e/d |
| | | | July 2012 | 20 | 0.8 | 2 | 4 | 2 | 8 | e/d180/120 |
| | | | February 2013 | 20 | 5 | 4 | 0.5 | 4 | 8 | high stress levels, needs |
| | | | | | | | | | | rtc |
| 224 | M | 1941 | June 2010 | 0 | 0 | n/a | 1 | n/a | 0.5 | |
| 28 | | | November 2011 | 0 | 0 | 2 | 10.5 | 2 | 8.5 | |
| | | | May 2012 | 20 | 5 | 2 | 9.5 | 2 | 8.5 | e/d/360/240 only took one |
| | | | | | | | | | | bottle, ret. spec. Said |
| | | | | | | | | | | “throw them away” |
| | | | December 2012 | 20 | 5 | 2 | 5 | 2 | 4.5 | e/d/360/240 |
| 686 | M | 1930 | October 2007 | 0 | 0 | n/a | 0 | n/a | 0 | vague decrease vision OD |
| 29 | | | July 2009 | 0 | 0 | 1 | 0 | 2 | 0 | |
| | | | June 2011 | 0 | 0 | 2 | 0 | 2 | 0 | OD moderate pucker, va |
| | | | | | | | | | | 20/40 |
| | | | December 2011 | 0 | 0 | 2 | 0 | 2 | 0 | OD moderate pucker, va |
| | | | | | | | | | | 20/40 |
| | | | June 2012 | 20 | 5 | 3 | 0 | 3 | 0 | OD mild pucker, va 20/25 |
| | | | December 2012 | 20 | 5 | 2 | 0 | 2 | 0 | OD mild pucker, va 20/25 |
| | | | April 2013 | 20 | 0.8 | 2 | 0 | 2 | 0 | |
| 689 | M | 1948 | July 2009 | 0 | 0 | | 9 | | | only has one eye |
| 30 | | | October 2009 | 5 | 0.25 | 2 | 10 | | | |
| | | | October 2009 | 5 | 0.25 | 2 | 10.5 | | | |
| | | | December 2009 | 10 | 0.5 | 4 | 10.5 | | | e/d/360/240 |
| | | | April 2012 | 5 | 0.25 | 4 | 11.5 | | | *plh 4 mm e/d/360/240 |
| | | | August 2012 | 20 | 5 | 3 | 10.5 | | | 4 mon. later*plh 2 mm, |
| | | | | | | | | | | e/d/360/240 |
| | | | February 2013 | 0 | 0 | 3 | 10.5 | | | |
| 763 | M | 1951 | August 2009 | 0 | 0 | 3 | 0 | 3 | 5.5 | |
| 31 | | | September 2010 | 0 | 0 | 3 | 0 | 4 | 5.5 | e/d/180/120 |
| | | | August 2012 | 5 | 0 | 4 | 0 | 4 | 0.5 | 23 mon later, inc |
| | | | | | | | | | | fish/chicken, dec beef |
| 150 | M | 1951 | February 2010 | 0 | 0 | 4 | 0 | 4 | 0 | |
| 32 | | | September 2011 | 0 | 0 | 4 | 11 | 4 | 0 | e/d/180/120 |
| | | | March 2012 | 20 | 5 | 3 | 5.5 | 3 | 0 | |
| | | | September 2012 | 20 | 5 | 3 | 5.5 | 3 | 0 | inc. BFV |
| 879 | F | 1946 | September 2010 | 0 | 0 | 4 | 0 | 4 | 0.5 | e/d/540/360 |
| 33 | | | September 2011 | 0 | 0 | 4 | 0 | 4 | 0.5 | e/d/180/120 |
| | | | March 2012 | 20 | 5 | 3 | 0 | 3 | 0 | e/d/360/240 |
| | | | September 2012 | 20 | 5 | 4 | 0 | 4 | 0 | e/d/360/240 |
| 131 | F | 1937 | January 2009 | 0 | 0 | 1 | 0.5 | 1 | 12 | |
| 34 | | | January 2010 | 0 | 0 | 2 | 0 | 1 | 13 | |
| | | | November 2010 | 0 | 0 | 2 | 0 | n/a | 15.5 | e/d/360/240 |
| | | | June 2011 | 10 | 2 | 3 | 0 | 2 | 15 | e/d/360/240 |
| | | | February 2012 | 10 | 2 | 2 | 0 | 2 | 6.4 | |
| | | | August 2012 | 10 | 2 | 2 | 0 | 2 | 3.8 | |
| 1774 | M | 1930 | June 2007 | 0 | 0 | | 0.5 | | 0 | next visit 41 mon. no |
| | | | | | | | | | | photos yet |
| 35 | | | November 2010 | 0 | 0 | 3 | 12 | 3 | 0 | IOL OU now |
| | | | June 2011 | 10 | 0 | 4 | 9.5 | 4 | 0 | e/d/180/120 |
| | | | August 2012 | 10 | 0 | 3 | 4.5 | 3 | 0 | 14 mon |
| | | | February 2013 | 0 | 0 | 3 | 4.5 | 4 | 0 | using garlic, cinnamon, |
| | | | | | | | | | | hot pepper, lemon, honey |
| 1662 | F | 1954 | September 2008 | 0 | 0 | | | | | od plh, no photos yet |
| 36 | | | October 2010 | 0 | 0 | 4 | 11.5 | 4 | 9 | od plh 8 × 5 mm. |
| | | | August 2012 | 5 | 1 | 3 | 11 | 3 | 6 | e/d/360/240 plh 8 × 5 mm |
| | | | February 2013 | 20 | 5 | 5 | 9 | 4 | 0 | |
| 630 | M | 1946 | June 2009 | 0 | 0 | 3 | 0 | 2 | 0 | 23 years diab. |
| 37 | | | June 2010 | 0 | 0 | 4 | 0 | 4 | 0.5 | |
| | | | June 2011 | 0 | 0 | 4+ | 0.5 | 4+ | 1 | |
| | | | July 2012 | 0 | 0 | 3 | 0 | 3 | 9 | |
| | | | March 2013 | 10 | 2.5 | 3 | 0 | 3 | 7 | worse, advised L 20 and |
| | | | | | | | | | | Z5 |
| 1010 | F | 1954 | January 2010 | 0 | 0 | 3 | 0 | 4 | 4.3 | OS “central red ‘scotoma’” |
| | | | | | | | | | | va 7.5- plh 10 mm |
| 38 | | | January 2012 | 0 | 0 | 3 | 0 | 4 | 8 | N/A plh mm |
| | | | July 2012 | 20 | 5 | 1 | 0 | 2 | 0 | Plh 5 mm |
| | | | March 2013 | 10 | 2.5 | 2 | 0 | 3 | 9 | worse, advised L 20 and |
| | | | | | | | | | | Z5 Plh 4 mm |
| 1011 | M | 1942 | November 2009 | 0 | 0 | 2 | 0 | 2 | 0 | Intermediate AMD OU |
| 39 | | | May 2010 | 10 | 2 | 3 | 0 | 3 | 0 | e/d/360/240 |
| | | | August 2010 | 10 | 2 | 4 | 10.5 | 4 | 2.5 | e/d/720/480 |
| | | | November 2010 | 10 | 2 | 3 | 9.5 | 3 | 2.5 | e/d/360/240 |
| | | | January 2013 | 20 | 5 | 4 | 8 | 3 | 2 | |
| | | | April 2013 | 20 | 5 | 2 | 4 | 3 | 1.5 | ERM resolving on 20/5, |
| | | | | | | | | | | AMD stable |
| 1151 | F | 1957 | January 2010 | 0 | 0 | 3 | 6.4 | 3 | 0 | od drusen and ERM |
| | | | | | | | | | | started L10, Z2 for drusen |
| 40 | | | July 2010 | 10 | 2 | 4 | 5.1 | 3 | 0 | e/d/360/240 |
| | | | January 2011 | 10 | 2 | 4 | 4.6 | 4 | 0 | e/d/360/240 |
| | | | July 2011 | 10 | 2 | 3 | 4.3 | 3 | 0 | e/d/360/240 |
| | | | February 2012 | 10 | 2 | 4 | 3.2 | 4 | 0 | ERM peeling off! |
| | | | | | | | | | | e/d/360/240 |
| | | | March 2012 | 10 | 2 | 4 | 2.7 | 4 | 0 | ERM peeling off! |
| | | | | | | | | | | e/d/360/240 |

Results: Treatment of retinal lesions with some combination of lutein and zeaxanthin was useful for decreasing subject's NDS's and ameliorating and decreasing the size of retinal lesions in subjects having VRT. Frequently, when patients stopped or reduced their dosage their NDS increased and ocular disease recurred. It is contemplated that unilaterally increased NDS and unilateral eye disease may be due to unilateral carotid stenosis.

Daily oral administration to a subject of 10-20 mg lutein and 0.5-5 mg zeaxanthin was sufficient to decrease NDS and retinal lesions in the majority of subjects who had not previously been treated with lutein and/or zeaxanthin. Some patients were on low doses of lutein and zeaxanthin for AMD and still developed VRTD. Increased doses of lutein and zeaxanthin were required to reverse VRTD.

### Example 3

### LZO3P Dosage Response Trials Indicate Positive Results for Treatment Ocular Health and Nutrient Deficiency as Indicated by Decreased Nutritional Deficiency Scores and Improvement in Various Ocular Symptoms

Subject Description: Data were collected from 41 subjects having various states of ocular health at a first time point and a second time point and optionally a third time point, the second time point being three months after the first and the third time point being three months after the second. Treatment was started after the first time point.

Treatment: Subjects were treated daily with the LZO3P composition (i.e., lutein (20 mg) zeaxanthin (5 mg), omega-3 fatty acids (180 mg) and DHA (120 mg)) following an evening meal comprising protein. Nutritional deficiency scores (NDS) and lesion sizes were measured prior to and following 3, and optionally 6, months of treatment. The nutritional deficiency scoring rubric used involved a scoring system of 1 to 5, wherein 1 indicates a healthy eye having sufficient nutrients and 5 indicates a severely nutrient deficient eye, wherein nutrient levels are low relative to a healthy eye and indicative of ocular disease or a likelihood of developing eye disease. In this study, subject ocular lutein and zeaxanthin levels were scored separately. In some instances a score for elevated retina and/or RNFL was included under the heading “3-D”. A 3-D score of 1 indicates normal 3-D appearance; 2 is moderately abnormal 3-D appearance; 3 is very abnormal 3-D appearance; 5 indicates the presence of a partial lamellar hole; and 8 indicates the presence of edema.

TABLE 2
Raw data collected from subjects during 3-month LZO3P trial. Separate lutein (L) and
zeaxanthin (Z) and 3-D ocular health score are provided for both eyes (OD and OS).

| Case | | | OD L | OD Z | OS L | OS Z | OD | OS | |
|---|---|---|---|---|---|---|---|---|---|
| # | gender | age | NDS | NDS | NDS | NDS | 3D | 3D | Comment |
| 2093 | 1 | 51 | 3 | 3 | 3 | 3 | Pre-erm | Pre-erm | |
| | | | 3.5 | 3 | 3.5 | 3 | Pre-erm | Pre-erm | Non-responder |
| 471 | 1 | 68 | 3 | 2 | 3 | 2 | 2 | 2 | OS rpe |
| | | | 2 | 1 | 4 | 3 | 2 | 2 | Early left carotid stenosis? |
| 2534 | 1 | 22 | 3 | 4 | 3 | 3 | pre-csr | pre-csr | No improvement in OS |
| | | | 2 | 3 | 3 | 3 | pre-csr | pre-csr | Early left carotid stenosis? |
| 387 | 1 | 58 | 4 | 3 | 4 | 3 | 1 | 1 | |
| | | | 3 | 2 | 3 | 2 | 1 | 1 | successful prevention |
| 470 | 1 | 67 | 2 | 3 | 4 | 3 | Od plh | OS pucker | |
| | | | 1 | 1 | 2 | 1 | 1 | 1 | mild left stenosis, od plh |
| | | | | | | | | | and OS pucker resolved in |
| | | | | | | | | | 3 months! |
| 1804 | 1 | 74 | 3 | 3 | 2 | 1 | 1 | 3 | OD iol, OU mild drusen & |
| | | | | | | | | | trace erm, OS macular |
| | | | | | | | | | edema |
| | | | 4 | 4 | 3 | 3 | 1 | 2 | OU drusen no change. |
| | | | | | | | | | Con'd Erm and edema |
| | | | | | | | | | gone. Needs M.D. referral |
| | | | | | | | | | for stenosis, maybe |
| | | | | | | | | | probiotics if -ve stenosis |
| 717 | 1 | 64 | 5 | 5 | 4 | 3 | 2 | 2 | od GA |
| | | | 2 | 2 | 2 | 2 | 3 | 3 | OD dec. fibrosis and dec. |
| | | | | | | | | | excavation of RNFL |
| 2366 | 1 | 85 | 3 | 3 | 2 | 3 | 2 | 2 | Ou AMD, OD micro plh |
| | | | 2 | 2 | 3 | 3 | 2 | 3 | 6 month trial, no L/Z/ |
| | | | | | | | | | omega previous 2 weeks, |
| | | | | | | | | | mild left stenosis |
| 2616 | 1 | 49 | 4 | 4 | 4 | 3 | 1 | 1 | |
| | | | 2 | 2 | 3 | 3 | 1 | 1 | successful prevention |
| 1492 | 2 | 46 | 3 | 3 | 2 | 2 | 1 | 1 | Diabetes, 1 micro |
| | | | | | | | | | aneurysm |
| | | | 2 | 2 | 3 | 3 | 1 | 1 | Early left carotid stenosis? |
| | | | | | | | | | 0 micro aneurysm |
| 526 | 1 | 64 | 4 | 4 | 4 | 4 | 2 | 2 | |
| | | | 2 | 2 | 2 | 2 | 1 | 1 | |
| | | | 3 | 3 | 3 | 3 | 2 | 2 | Recent increased stress in |
| | | | | | | | | | severely stressful life |
| | | | | | | | | | situations |
| 2495 | 2 | 65 | 5 | 5 | 3 | 3 | 2 | 2 | OD small drusen |
| | | | 4.5 | 4.5 | 2.5 | 2.5 | 1 | 1 | right stenosis and needs |
| | | | | | | | | | probiotics? (slow |
| | | | | | | | | | response) |
| | | | 4 | 4 | 2 | 2 | 1 | 1 | |
| 2169 | 2 | 55 | 3 | 3 | 3 | 3 | 3 | 3 | |
| | | | 2 | 2 | 3 | 3 | 2 | 2 | |
| | | | 2 | 3 | 2 | 2 | 2 | 2 | successful prevention |
| 2266 | 1 | 58 | 3 | 3 | 3 | 3 | 2 | 2 | |
| | | | 2 | 2 | 2 | 2 | 1 | 2 | successful prevention |
| 2169 | 2 | 55 | 3 | 3 | 3 | 3 | 3 | 3 | |
| | | | 2 | 2 | 3 | 3 | 2 | 2 | |
| | | | 2 | 2 | 2 | 2 | 1 | 2 | successful prevention |
| 2643 | 1 | 60 | 3 | 3 | 4 | 3 | 1 | 1 | |
| | | | 2 | 1 | 2 | 1 | 1 | 1 | successful prevention, 3-d |
| | | | | | | | | | retina layer now flat OU |
| 1011 | 1 | 70 | 4 | 2 | 4 | 2 | 1 | 1 | OU AMD, OD pucker, L |
| | | | | | | | | | 10, Z 2 before trials |
| | | | 3 | 1 | 3 | 2 | 1 | 1 | |
| | | | 2 | 1 | 2 | 2 | 1 | 1 | pucker gone, AMD same |
| 1081 | 1 | 68 | 3.5 | 2 | 3 | 2 | 1 | 1 | |
| | | | 2 | 1 | 2 | 1 | 1 | 1 | successful prevention, 3-d |
| | | | | | | | | | retina layer now flat OU |
| 747 | 1 | 62 | 3 | 3 | 3 | 3 | 3 | 3 | |
| | | | 1 | 1 | 1 | 1 | 1 | 2 | successful prevention |
| 2161 | 2 | 53 | 4 | 3 | 3 | 2 | 2 | 1 | OD amblyopia, OS |
| | | | | | | | | | macular drusen, L 10, Z 2 |
| | | | | | | | | | before trials |
| | | | 3.5 | 2 | 4 | 2 | 2 | 1 | |
| | | | 3 | 2 | 3 | 2 | 1 | 2 | slow improvement, OS |
| | | | | | | | | | drusen no change |
| 481 | 1 | 74 | 4 | 2 | 4 | 2 | 1 | 1 | |
| | | | 2 | 2 | 2 | 2 | 1 | 2 | successful prevention |
| 1202 | 2 | 73 | 4 | 2 | 3.5 | 2 | 1 | 1 | |
| | | | 2 | 2 | 2 | 2 | 1 | 2 | successful prevention |
| 1441 | 1 | 49 | 3 | 3 | 3 | 2 | 3 | 3 | |
| | | | 2.5 | 3 | 3.5 | 3 | 3 | 3 | did not use 180/120, Early |
| | | | | | | | | | left carotid stenosis? |
| 93 | 1 | 64 | 2 | 2 | 4 | 4 | 1 | 1 | od drusen |
| | | | 3 | 3 | 3 | 3 | 1 | 1 | right stenosis |
| 718 | 1 | 75 | 3 | 2 | 2 | 4 | 1 | 2 | June 2010 OS (erm) |
| | | | | | | | | | vitrectomy, IOL, OU small |
| | | | | | | | | | plh & severe dry eye, |
| | | | 2 | 1 | 3 | 3 | 1 | 2 | 5 month trial, OU plh |
| | | | | | | | | | smaller & eyelid margins |
| | | | | | | | | | no longer red, left |
| | | | | | | | | | stenosis? (mid-November 2012 |
| | | | | | | | | | $1500 heat and squeeze |
| | | | | | | | | | eyelid therapy) |
| 2411 | 1 | 44 | 3 | 3 | 2 | 2 | 2 | 2 | sig. GI probs. Also took |
| | | | | | | | | | probiotics |
| | | | 2 | 2 | 3 | 3 | 2 | 2 | lactose intolerance gone, |
| | | | | | | | | | left stenosis? Reg. Bms, |
| | | | | | | | | | no gas, inc. energy. |
| 1784 | 2 | 62 | 3 | 3 | 2 | 3 | 3 | 8 | |
| | | | 2 | 2 | 3 | 4 | 2 | 3 | left stenosis? OS edema |
| | | | | | | | | | gone |
| 2595 | 1 | 41 | 2 | 2 | 2 | 2 | 2 | 8 | OU Pre-csr |
| | | | 3 | 4 | 3 | 3 | 3 | 3 | OS edema gone |
| 1431 | 1 | 75 | 3 | 3 | 3 | 3 | 1 | 1 | OU AMD & mac. Nevi |
| | | | | | | | | | (nevi, no change × 3 |
| | | | | | | | | | years) |
| | | | 5 | 5 | 5 | 5 | 1 | 1 | OU stenosis. Referred to |
| | | | | | | | | | MD |
| 1432 | 2 | 80 | 2 | 2 | 2 | 2 | 1 | 8 | OS edema |
| | | | 2 | 2 | 1 | 1 | 1 | 1 | OS edema gone |
| 459 | 2 | 64 | 4 | 4 | 4 | 3 | 1 | 2 | OD amblyopia, OS |
| | | | | | | | | | macular drusen, L 10, Z 2 |
| | | | | | | | | | before trials |
| | | | 2 | 2 | 3 | 2 | 1 | 2 | successful prevention |
| 353 | 1 | 79 | 2 | 2 | 2 | 2 | 2 | 3 | OS erm, 7 month trial |
| | | | 3 | 2 | 3 | 2 | 1 | 2 | probiotics added 5 month |
| | | | | | | | | | trial |
| | | | 2 | 2 | 3.5 | 2 | 1 | 2 | no change OS erm, left |
| | | | | | | | | | stenosis? |
| 615 | 1 | 68 | 4 | 3 | 4 | 3 | 3 | 1 | |
| | | | 2 | 2 | 2 | 2 | 1 | 1 | successful prevention |
| 1456 | 2 | 69 | 4 | 4 | 3 | 3 | 8 | 2 | OU iol, OD RNFL edema |
| | | | 3 | 3 | 2 | 2 | 8 | 1 | OS cws, refer to MD for |
| | | | | | | | | | hypertension |
| 2578 | 2 | 67 | 3 | 2 | 3 | 2 | 1 | 1 | |
| | | | 2 | 2 | 2 | 2 | 1 | 1 | successful prevention |
| 2579 | 2 | 66 | 3 | 2 | 3 | 2 | 1 | 1 | |
| | | | 3 | 2 | 3 | 2 | 1 | 1 | Non-responder |
| 2542 | 1 | 50 | 4 | 4 | 3 | 4 | 3 | 3 | probiotics also |
| | | | 3 | 2 | 4 | 3 | 2 | 2 | GI more regular, watch OS L |
| 865 | 1 | 71 | 3 | 2 | 4 | 3 | 1 | 3 | arcade ends poorly |
| | | | | | | | | | perfused |
| | | | 3 | 2 | 4 | 2 | 1 | 1 | only OS L and 3-d |
| | | | | | | | | | improved |
| 2562 | 1 | 70 | 3.5 | 4 | 3 | 1 | 3 | 1 | OD iol |
| | | | 3 | 3 | 2 | 1 | 1 | 1 | “better distance vision” |
| 955 | 2 | 60 | 4 | 3 | 4 | 3 | 1 | 1 | sig. GI probs. Also took |
| | | | | | | | | | probiotics |
| | | | 3 | 2 | 2 | 1 | 1 | 1 | Improved facial rosacea, |
| | | | | | | | | | virus recovery now 2-3 |
| | | | | | | | | | weeks instead of 4 weeks |
| 78 | 1 | 71 | 4 | 3 | 4 | 3 | 1 | 5 | OU erm, OS plh |
| | | | 2 | 1 | 2 | 1 | 1 | 1 | Alzheimer's no change |

Results: Treatment of subject with LZO3P was useful for decreasing subject's NDSs (Table 2). In some cases, Lutein NDS and Zeaxanthin NDS differ in the same eye (Table 2). In a review of 8 patients from table 1, zeaxanthin NDSs vs VRT reversal shows that 0.8 zeaxanthin, “Z”, was not enough to reverse VRT or to substantially improve Z NDSs. An amount of about 5.0 mg of Z is preferred to reverse microvascular non-perfusion and VRT and to improve Z ND scores. More rapid and greater improvement (reduction in lesion size) occurred when Z was 5.0 mg.

### Example 4

### Treatment of Subjects Having Lutein and Zeaxanthin Deficiencies with LZO3P Can Improve NDS and Glaucoma

Low tension glaucoma (LTG) is a condition wherein optic nerve head (ONH) degeneration develops despite normal intraocular pressure. It is contemplated that LTG occurs due to MVNP of the ONH. In some cases, lutein and the zeaxanthin NDS (Z NDS) differ in the same eye. In a review of 8 patients from table 1, 5.0 mg of Z was found to reverse microvascular non-perfusion and VRT and to improve Z NDS.

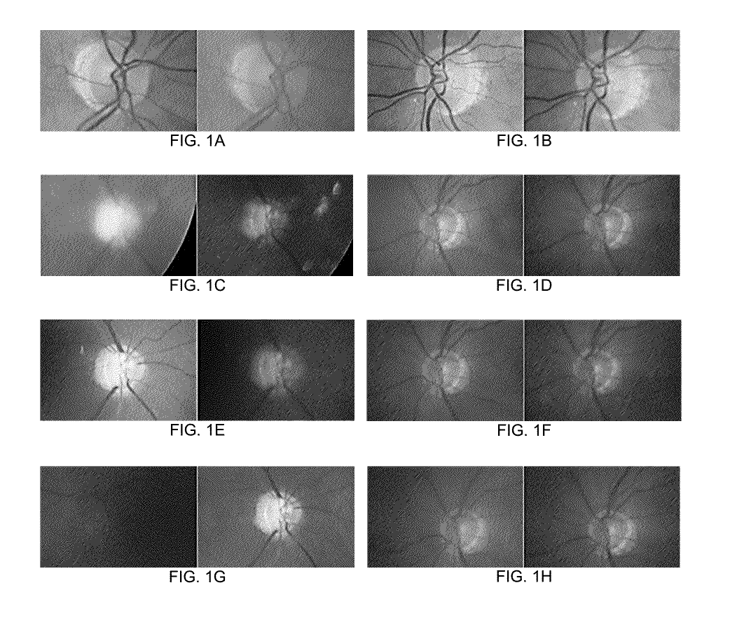

Subject Description: Patient 2740 is an 85 year old female. Upon examination on Apr. 29, 2013, the patient had a lutein NDS of 3+ and a zeaxanthin NDS of 5+ in both eyes. It is contemplated that the poor Z NDS is correlated with poor vascular perfusion. OD 10P (intraocular pressure) 14 CCT (central corneal thickness) 516, normal cupping (FIG. 1A); OS IOP 14 CCT 521, inferior notching, probable early LTG (FIG. 1B).

Treatment 1: Additional glaucoma testing recommended and LZO3P therapy started.

Subject Description: Patient 1061 is a 67 year old male who had asymmetric chronic glaucoma. Prior to LZO3P treatment, on Jun. 3, 2011, patient 1061 had an OD NDS of 5+, retinal detachment, intraocular lens, and end stage glaucoma with severe glaucomatous loss (FIG. 1C). Patient's OS had an average NDS of 3 with moderate and stable glaucomatous loss (FIG. 1D).

Prior to treatment, on Sep. 21, 2012 the subject's inferior OS cupping had increased as did OS NDS. OD had an NDS of 5+ (FIG. 1E) and OS had an NDS of 4 (FIG. 1F).

Treatment 2: LZO3P daily treatment (patient was receiving standard glaucoma treatment as well).

Following 5-months LZO3P treatment, glaucoma and nutrient deficiency in the patient's OD remained severe (NDS=4) (FIG. 1G). However, OS cupping appeared to be stabilizing and OS NDS had improved to 2 (FIG. 1H). It is hypothesized that the NDS improvement observed in the patient's OS indicated reversal of end organ microvascular non-perfusion and stabilization of ocular disease.

### Example 5

### LZO3P Treatment of Subjects Having Lutein and Zeaxanthin Deficiencies Can Improve Geographic Atrophy

Geographic Atrophy: patients having end stage AMD, wherein all three layers of the retina scar and/or disappear.

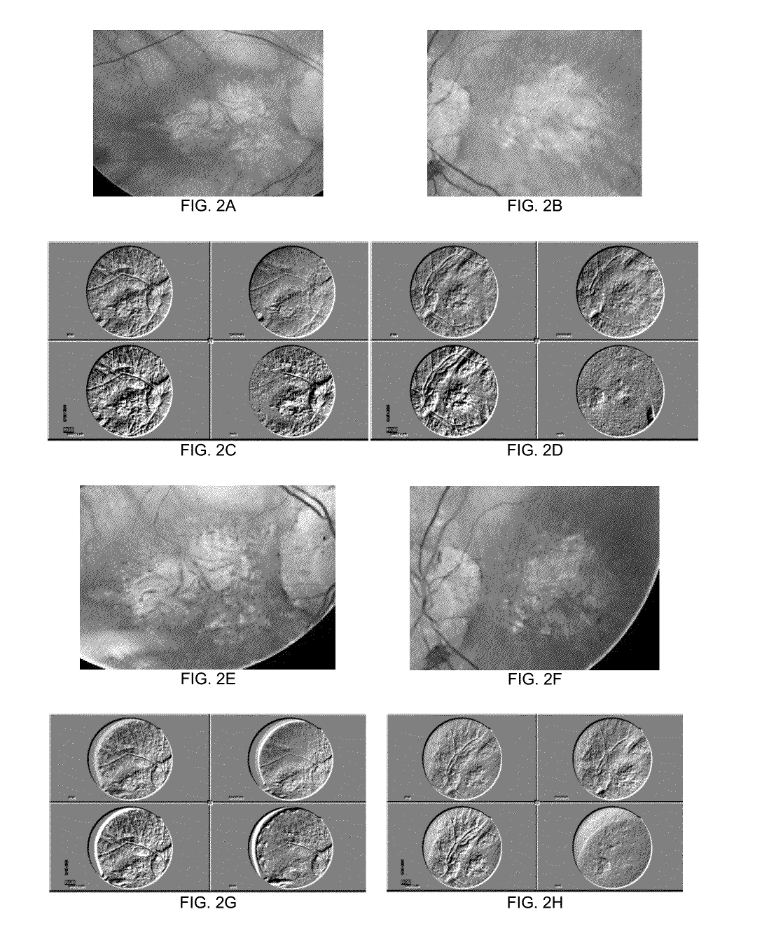

Subject Description: Patient 252 is an 86 year old female who had fibrosis, excavation and VA LP OU in both eyes (i.e., vision comprising only light perception; lacking detailed vision). It is contemplated that this patient had severe MVNP in both eyes. On Nov. 19, 2012 the patient had an OU NDS of 5 (OD before and after treatment, FIGS. 2A and C; OS before and after treatment, FIGS. 2B and D).

Treatment: 4 months of continued LZO3P therapy.

Results: Upon examination on Mar. 18, 2013, following four months LZO3P treatment, the patient had an OU NDS of 3, choroidal blood vessels had increased in diameter, fibrosis reversal was observed, excavation was reduced and it is contemplated that MVNP had reversed (OD before and after treatment, FIGS. 2E and G; OS before and after treatment, FIGS. 2F and H).

### Example 6

### LZO3P Treatment of Subjects Having Lutein and Zeaxanthin Deficiencies Can Improve Diabetic Retinal Fibrosis

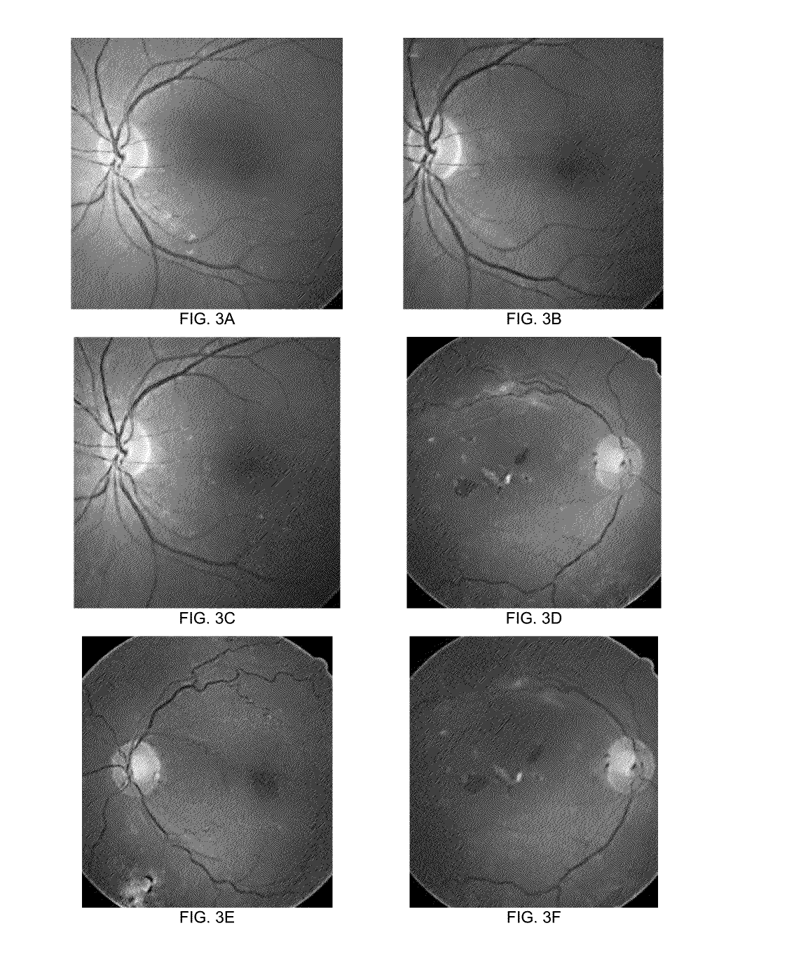

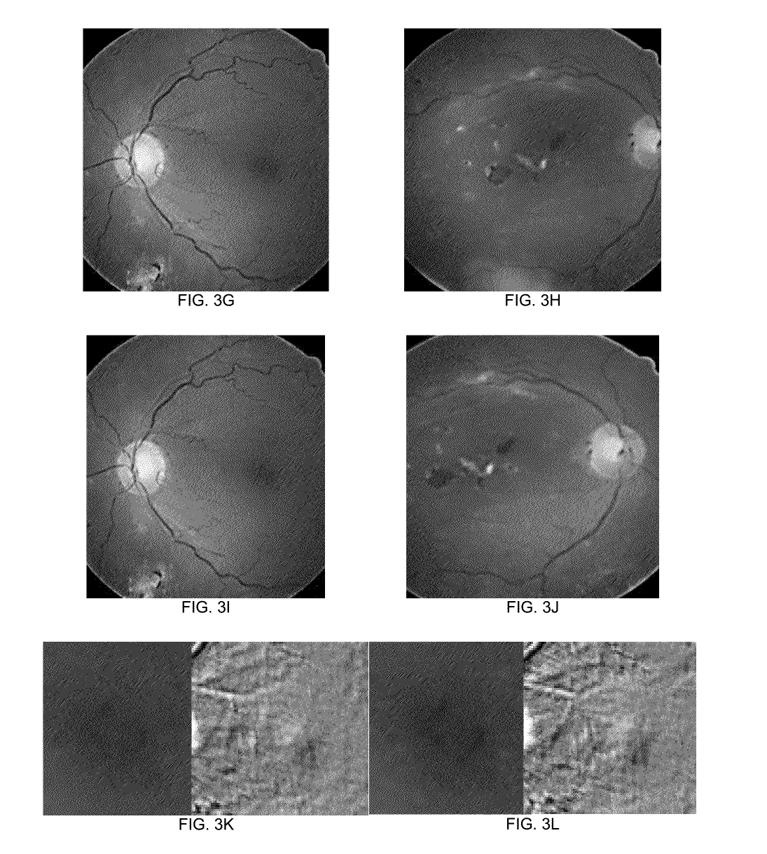

Subject Description: Patient 1010 is a 66 year old female who has had diabetes for 23 years. Upon examination on Jan. 26, 2012 the patient exhibited mild retina RNFL fibrosis of the left eye inferior arcade (FIG. 3A)

Treatment 1: Lutein (20 mg) and zeaxanthin (5 mg) daily.

Results: Following six months treatment, the patient's mild retina/RNFL and fibrosis were reversed (FIG. 3B).

Treatment 2: Lutein (20 mg) and zeaxanthin (5 mg) every other day.

Results: Following 6 months on lutein 20 mg and zeaxanthin 5 mg every other day, mild retina and RNFL fibrosis returned (FIG. 3C).

Subject Description: Patient 1625, a 79 year old male with diabetes for 25 years, suffered kidney failure and 6 resuscitations in 2011. Upon examination on Jun. 30, 2011, the patient's OD exhibited severe RNFL fibrosis and had an OD NDS of 4 (FIG. 3D) and the patient's OS exhibited moderate RNFL fibrosis, partial lamellar hole and had an NDS of 5 (FIG. 3F).

Treatment: Daily LZO3P treatment.

Results: Upon examination on Apr. 23, 2012, following 10 months treatment, the severe OD fibrosis was not reversed despite treatment (FIG. 3F), however the patient's OD NDS had been reduced to 2 (FIG. 3F). RNFL fibrosis was improved in the patient's OS (FIG. 3G) and the patient's OS NDS had been reduced to 3 (FIG. 3G).

Following 17 months daily LZO3P treatment (Dec. 5, 2012), the patient exhibited an OD NDS of 2 (FIG. 3H) and OS RNFL fibrosis with an NDS of 4 (FIG. 3I). Given the increase of the patient's OS NDS and lack of improvement in the patient's OS fibrosis, it is contemplated that the patient had developed stenosis of his left carotid artery.

Upon examination on Jun. 3, 2013, following an additional 6 months of daily LZO3P therapy, the patient's OD had an NDS of 2 (FIG. 3J) and an OS NDS of 3 (FIGS. 3K and L). Further, the patient's OS probable left carotid stenosis appeared to be reduced and the OS partial lamellar hole had improved along with improved visual acuity to 20/30.

Best corrected visual acuity, BCVA, is a measure of ocular health. BCVA of 20/50 is required to obtain a driver's license in many jurisdictions.

Date/OD BCVA/OS BCVA

June 2011/20/70/20/40

April 2012/20/70/20/40

December 2012/20/70/0/40-

June 2013/20/60/20/30

### Example 7

### Nutrient Treatment of Subjects Having Lutein and Zeaxanthin Deficiencies Can Improve Retinal RNFL Fibrosis

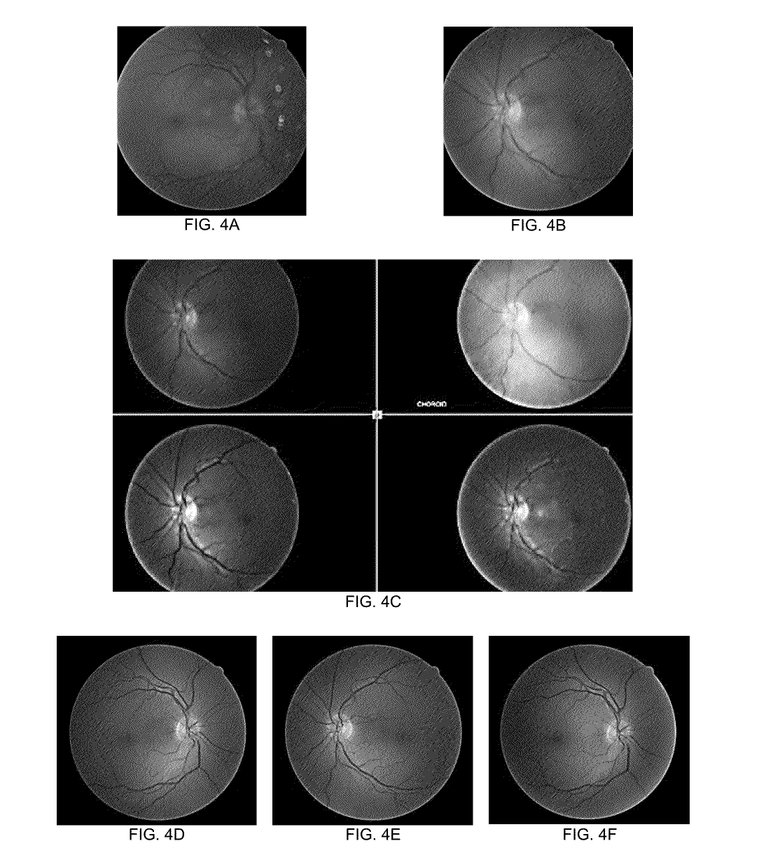

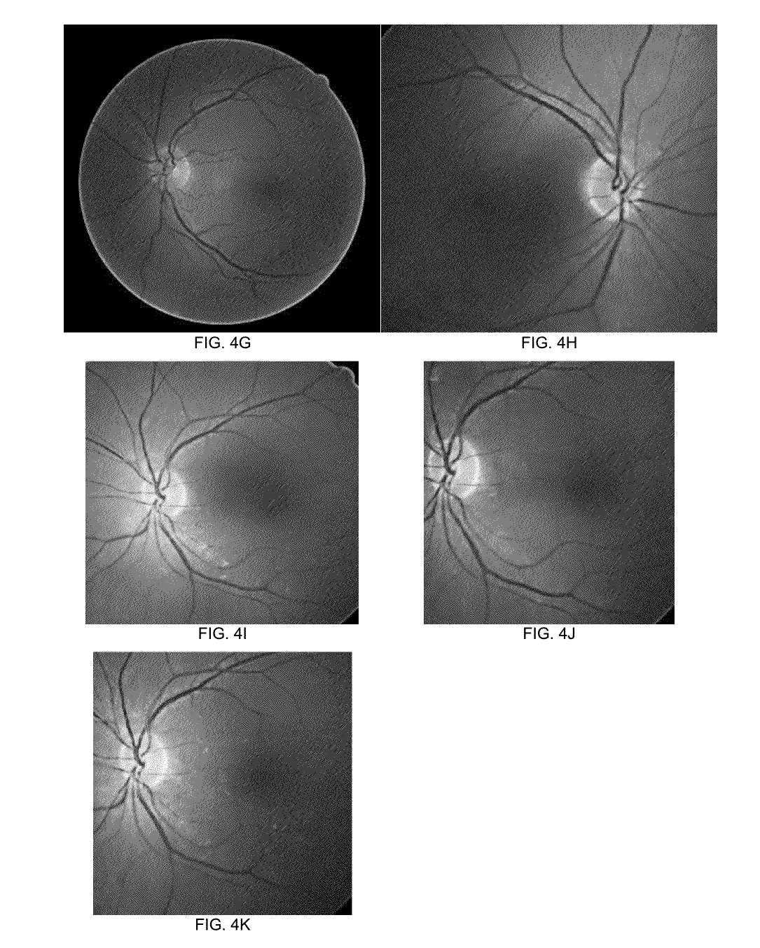

Subject Description: Patient 1548 is a 54 year old male who had a history of heavy drinking (40 ounces hard liquor daily) until 2009. Upon examination on Jul. 26, 2010, while the patient was taking lutein (5 mg) and zeaxanthin (0.25 mg daily), the patient had an OD NDS of 3 (FIG. 4A) and an OS NDS of 5 (FIG. 4B) accompanied by severe OS retinal-RNFL fibrosis (FIG. 4C). It is contemplated that the patient also had stenosis of the left carotid artery, correlated with the severe fibrosis and poor NDS.

Treatment 1: Lutein (5 mg) daily and fish five times per week for over 24 months.

Results: Upon examination of Nov. 13, 2012, the patient had an OU NDS of 4 (FIGS. 4D and E).

Treatment 2: LZO3P daily.

Results: Upon examination on Jun. 4, 2013, the patient's fibrosis had resolved and he had an OU NDS of 3 (FIGS. 4F and G), indicating that the patient's risk for chronic disease had been reduced following LZO3P treatment.

### Example 8

### Nutrient Treatment of Subjects Having Lutein and Zeaxanthin Deficiencies Can Improve Wet AMD

Wet AMD occurs when ischemic retinal tissue secretes vascular endothelial growth factor (VEGF), which causes growth of fragile new blood vessels that leak fluid and blood. This edema and hemorrhaging damages retinal tissue and reduces vision. Intra-vitreal injection of anti-VegF compositions is the current standard of care for wet AMD. However, it is contemplated that such injections do not treat the ischemia that is contemplated to underlie wet AMD.

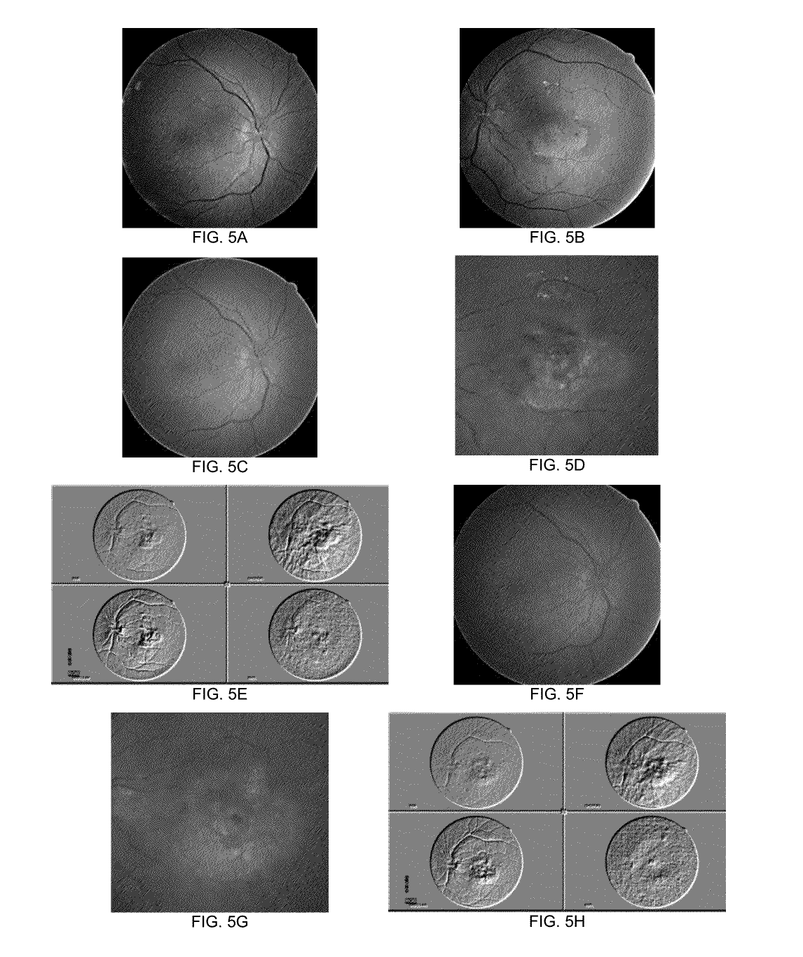

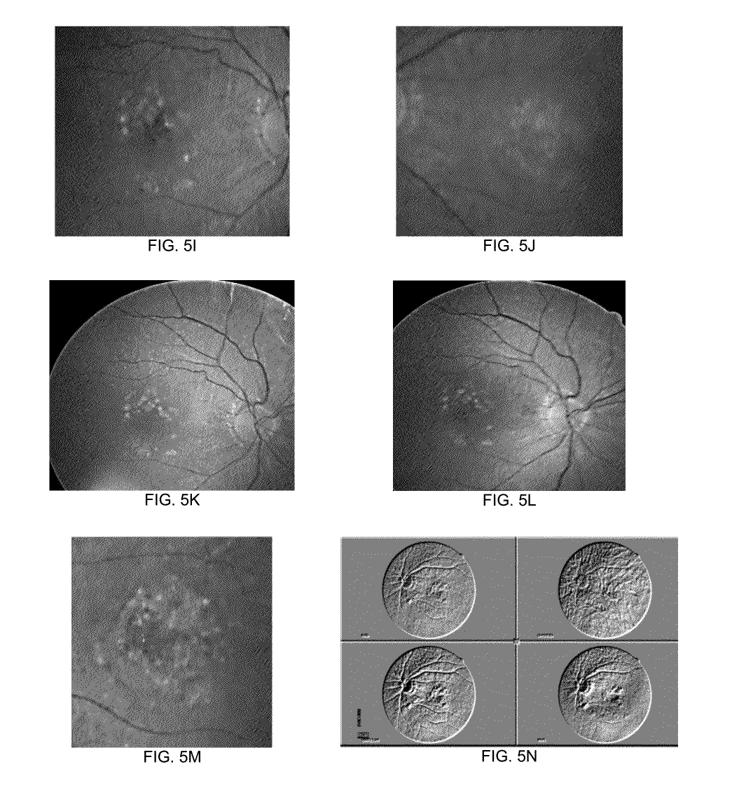

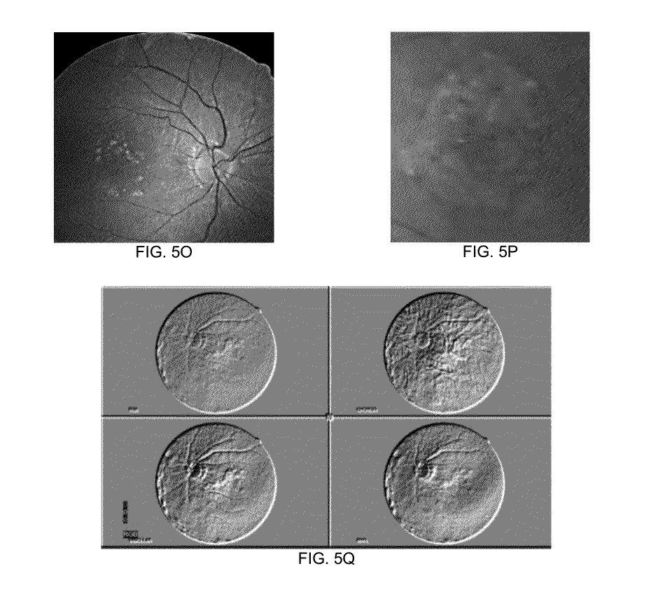

Subject Description: Patient 610 is a 70 year old male who was found to have an OD NDS of 2-3 and dry AMD (FIG. 5A) and an OS NDS of 4-5 and wet AMD (FIG. 5B) upon examination on Jun. 4, 2009. The patient was referred to retinal specialist who did not prescribe eye nutrient supplementation, but prescribed monthly intravitreal anti-VEGF injections into his left eye. Upon examination on Mar. 4, 2011, the patient had an OD NDS of 3 and dry AMD (FIG. 5C) and an OS NDS of 5 (FIG. 5D), OS geographic atrophy and ERM (FIG. 5E).

Treatment: One Vitalux plus omega™ pill twice daily(total daily dose of 5 mg lutein, 1 mg zeaxanthin, 200 mg EPA and 100 mg DHA).

Results: Upon examination on Nov. 6, 2012, the patient had an OD NDS of 5 and his OD dry AMD had worsened (FIG. 5F). However, the patient had an OS NDS of 3 (FIG. 5G), and the OS choroidal nonperfusion and GA were reversing and the OS ERM was absent (FIG. 5H). It is contemplated that the failure of the OD improvement is associated with carotid stenosis. It is contemplated that the failure of the OD improvement is associated with carotid stenosis of the right CCA and/or ICA. This low dose of lutein and zeaxanthin is not enough to prevent or reverse carotid artery stenosis.

Subject Description: Patient 2363 is a 93 year old female who had a history of wet AMD and intravitreal injections in her OS. Upon examination on Apr. 13, 2012, the patient was taking 2 Vitalux Plus Omega™ pills twice daily with meals Total daily dose of lutein, 10 mg, zeaxanthin, 2 mg, EPA 400 mg, DHA 200 mg, plus other antioxidants. Despite this “standard of care” nutrient therapy, the patient was developing early wet AMD in her OD. The patient's OS had macular scaring and GA and an OS NDS of 3 (FIG. 5I) and an OD with wet AMD and an NDS of 5 (FIG. 5J). It is contemplated that the patient had a right carotid stenosis. She was referred to a retinal specialist.

Upon examination on Oct. 5, 2012, the patient had received an intravitreal injection in her right eye in August 2012 (FIG. 5K).

Treatment: 2 Vitalux plus omega twice daily with meals and 20 mg lutein and 5 mg zeaxanthin with supper.

Results: Upon examination on Feb. 5, 2013, the patient had an OD NDS of 5 (FIG. 5L) and her OD had macular microheme (FIG. 5M). The patient had an OS NDS of 3 (FIG. 5N).

Upon examination on Apr. 30, 2013, the patient had an OD NDS of 3 (FIG. 5O) and the OD macular microheme was absent (FIG. 5P). The patient had an OS NDS of 3

(FIG. 5Q). The patient's probable right carotid stenosis was resolving and it was contemplated that full vascular perfusion OU should prevent recurrent wet AMD OU.

Unfortunately, miscommunication resulted in the patient reverting back to 2 Vitalux plus Omega™ pills, twice daily with meals. She was seen August, 2013 with an episode of total black out of vision in her right eye for 24+ hours. Her OD NDS was again 5, confirming a right carotid stenosis that caused her temporary right eye stroke.

Discussion: Results of the AREDS 2 study, released June 2013, indicate that 10 mg lutein, 2 mg zeaxanthin and antioxidants is not a combination that is adequate to treat moderate to severe eye disease. This LZO3P study and the four year study of VRT patients concur. (table 1 and table 2) It is contemplated that an advanced stage of carotid arterial stenosis is present on the same side(s) of the head as moderate to severe eye disease. Daily doses of at least 20 mg lutein and 5 mg of zeaxanthin are required to treat advanced carotid arterial sclerosis.

Measuring the lutein and zeaxanthin pigment density for the entire posterior pole of the eye, using the methods disclosed herein provides a complete geographic measurement that demonstrates the areas of the eye that are deficient in lutein and the areas of the eye that are deficient in zeaxanthin. Lutein deficiency is seldom the same level as zeaxanthin deficiency. It is contemplated that lutein and zeaxanthin levels reflect: i) the level of blood supply to each eye; ii) the level of absorption of lutein and zeaxanthin from the intestine; and iii) the level of consumption of lutein and zeaxanthin rich foods or supplements.

### Example 9

### Nutrient Treatment of Subjects Having Lutein and Zeaxanthin Deficiencies Can Improve Vitreoretinal Traction Disorders

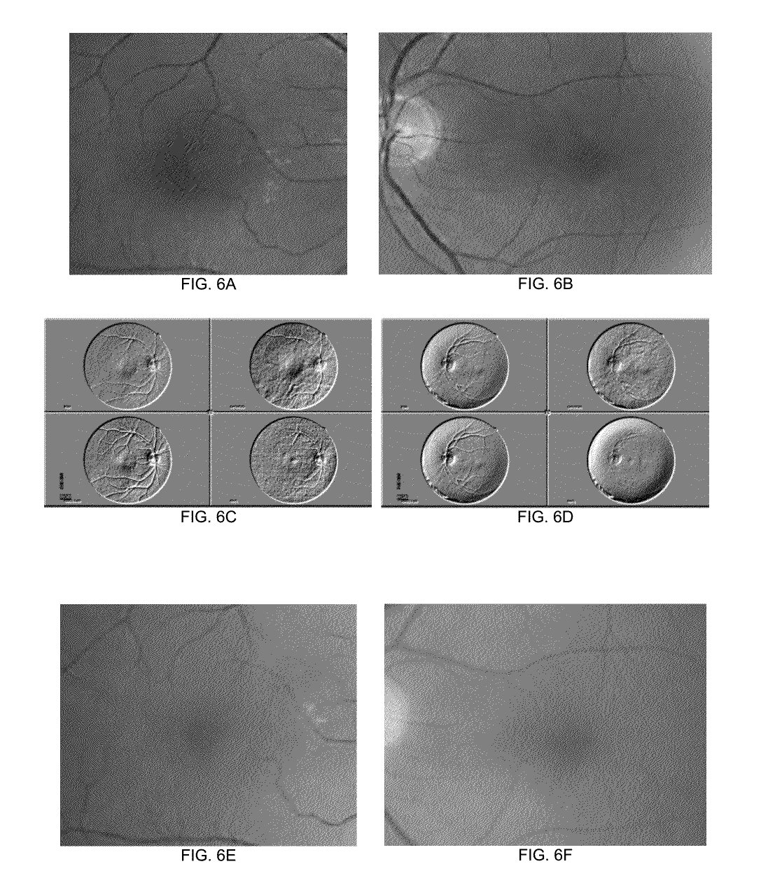

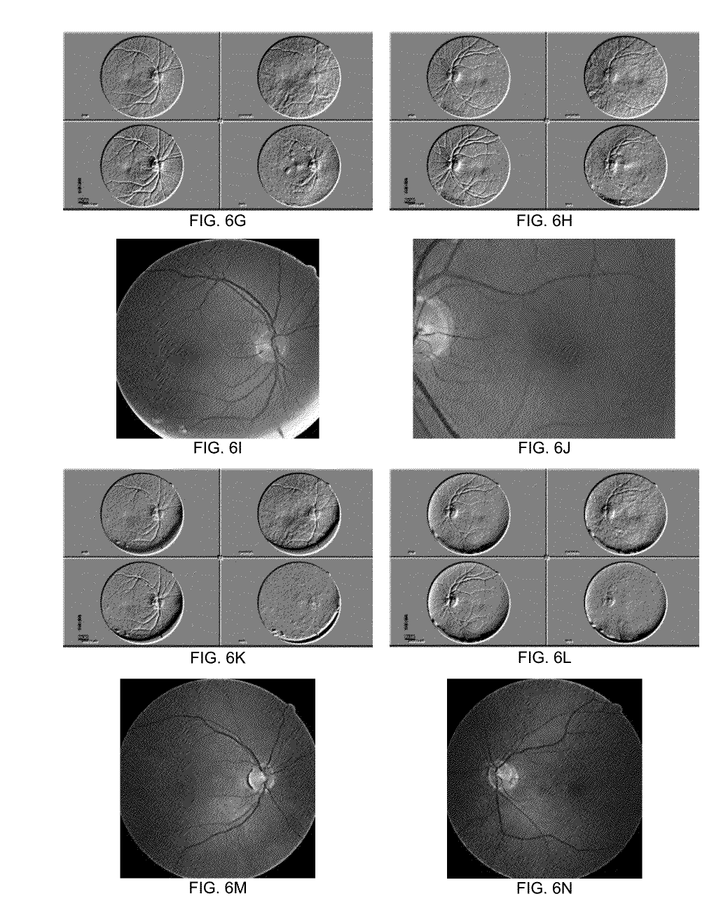

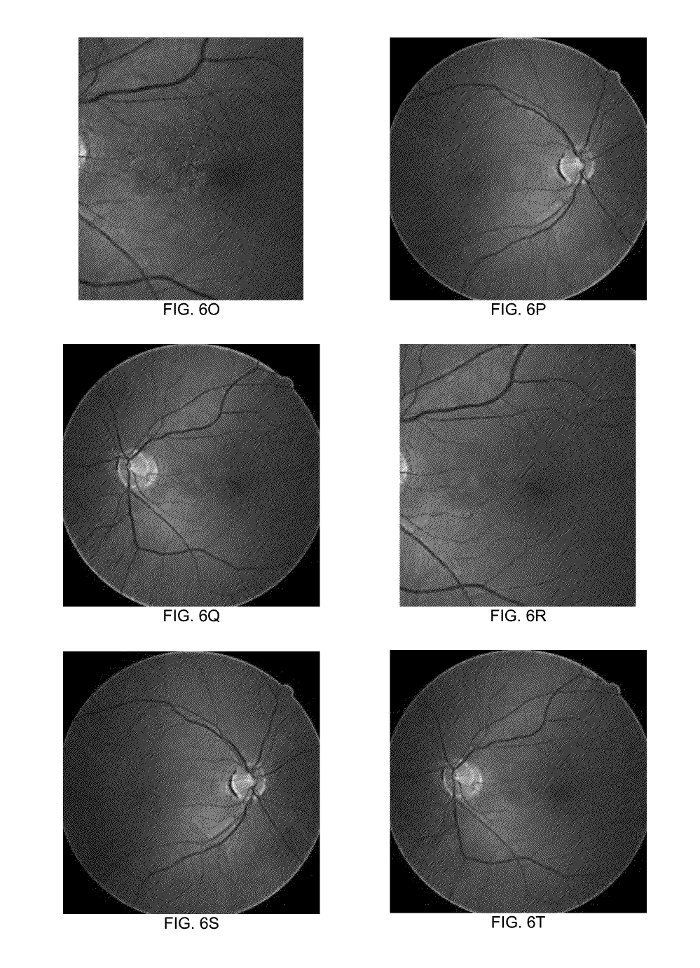

Subject Description: Patient 730, a 74 year old male, upon examination in November 2010, the patient had an OD NDS of 3 (FIG. 6A) and an OS NDS of 4 (FIG. 6B). The patient had an OD central macular edema (CME) (FIG. 6C) and an OS macular pucker (FIG. 6D).

Treatment: Lutein (10 mg) and zeaxanthin (2 mg) daily.

Results: Upon examination in November 2011, the patient had an OD NDS of 2 (FIGS. 6E and G) and an OS NDS of 2 (FIGS. 6F and H) and macular pucker had improved.

Treatment: Lutein (20 mg) and zeaxanthin (5 mg) daily.

Results: Upon examination in May 2012, following 6 months of treatment, reversal of CME (FIGS. 6I and K) and macular pucker (FIGS. 6J and L) were evident and the patient had an OU NDS of 2.

Subject Description: Patient 402 is a 54 year old female who, upon initial examination in April 2009, the patient had an OD NDS of 4 (FIG. 6M) and an OS NDS of 4 (FIG. 6N). The patient had an OS ERM (FIG. 6O).

Treatment: Lutein (20 mg) and zeaxanthin (5 mg) daily.

Results: Upon examination in November 2010, the patient had an OU NDS of 3 (FIGS. 6P and Q). The patient's OS ERM was reversed (FIG. 6R).

Treatment 2: Lutein (20 mg) and zeaxanthin (5 mg) daily.

Results: Upon examination in March 2012, the patient had an OD NDS of 4 (FIG. 6S) and an OS NDS of 3 (FIG. 6T). The OD NDS demonstrated reduced perfusion in the vascular arcades of the right eye. It is contemplated that this is due to early right carotid stenosis. It is contemplated that this may increase the patient's risk of right CRVO or BRVO.

### Example 10

### LZO3P Treatment of Subjects Having Lutein and Zeaxanthin Deficiencies Can Prevent BRVO

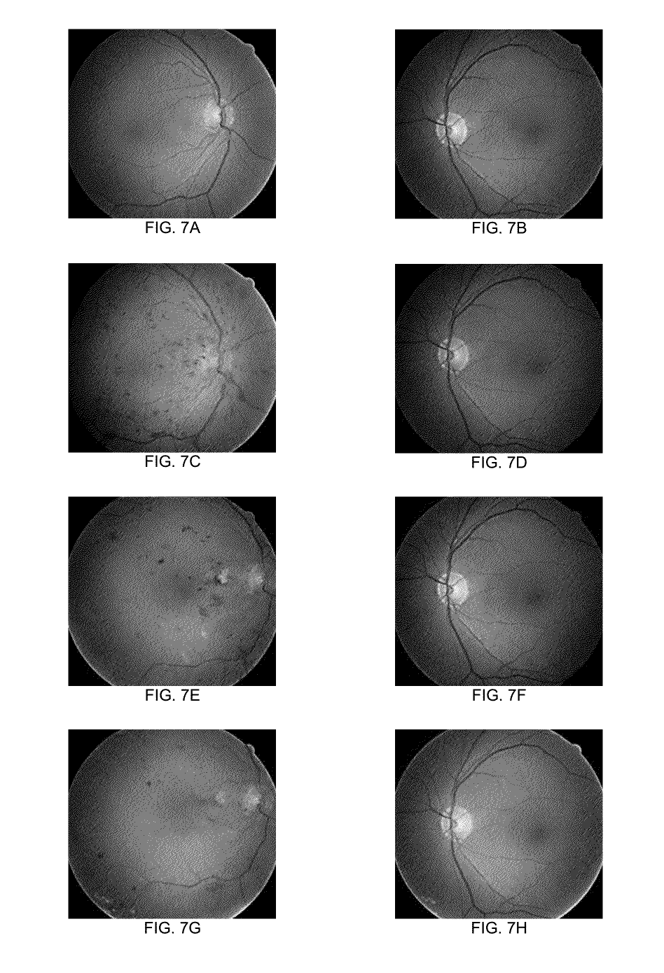

Subject Description: Patient 759 is a 75 year old male. He became diabetic in 1994. Eye exams between 2001 and 2007 were normal and the patient's VA was 20/20 in each eye. In 2008, his right eye had developed mild diabetic retinopathy and VA decreased to 20/30 in each eye. June 2009, he had heart bypass surgery. Upon examination on August 2009, he had mild diabetic retinopathy in both eyes and NDS was 3.5 OU (FIGS. 7A and B).

Upon examination on Aug. 10, 2010, the patient had early OD CRVO (FIG. 7C). NDS cannot be measured in acute BRVO; therefore, OD NDS is not reported. The patient's OS had an NDS of 4 (FIG. 7D). Patient's VA was 20/25 in each eye.

The patient received Lucentis intravitreal injections monthly from August 2010 through August 2011 and injections every 6 weeks thereafter. Upon examination on Sep. 8, 2011, the patient's OD had a VA of 20/400 (only scanning vision) (FIG. 7E) and an OS of 20/40 (FIG. 7F). The retinal arteries of the posterior pole were almost completely blocked. The right eye had end-stage microvascular non-perfusion of the posterior pole retina. His left eye arteries remained perfused and usable vision remained of 20/40.

Upon examination of Mar. 8, 2012, the patient's OD had a VA of 20/400 with central scotoma (FIG. 7G) and an OS VA of 20/40 (FIG. 7H).

Injections continued to September 2012, at which time OD VA was CF at 10 feet. (CF=count fingers). The inferior artery was now a white ghost vessel. Patient was prescribed LZO3P. Injections continued and patient developed severe pain and redness (presumed endophthalmitis).

May 2013 exam, OD massive hemorrhaging was present (FIG. 7F). OS VA was mildly improved to 20/30+.

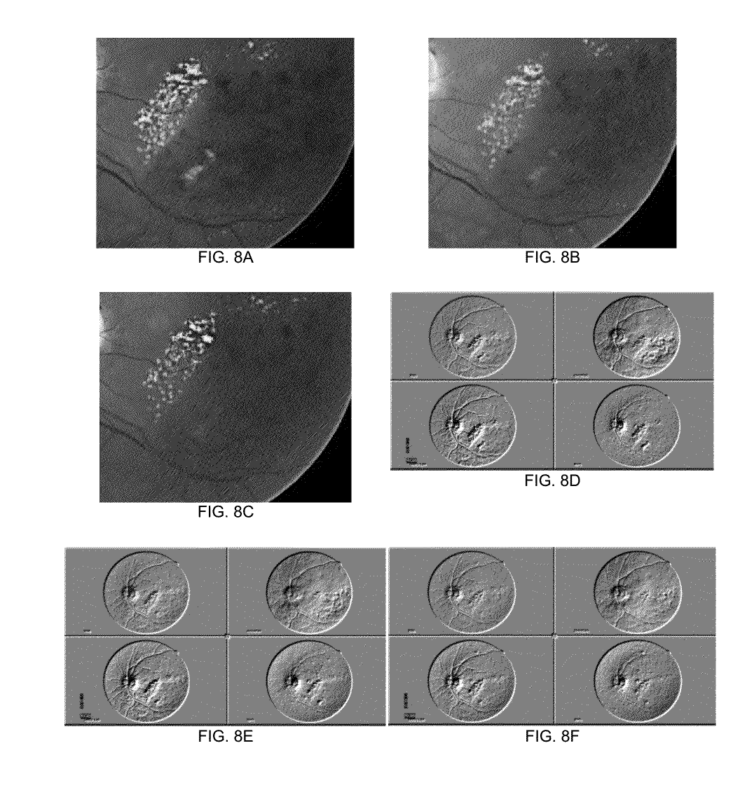

Subject Description: Patient 272 is a 47 year old male who was examined on Mar. 9, 2009 and May 10, 2012. At both of those visits VA was 20/20 in each eye and NDS was 3.5 in each eye (FIG. 8A).

Upon examination on Mar. 25, 2013, patient had a left branch retinal vein occlusion and BRVO that was likely at least 6 months old. He had an OU NDS of 3.5 and a visual acuity of OD 20/20 and OS 20/200. OD VA remained 20/20 throughout the following treatment period.

Treatment: LZO3P daily.

Results: Upon examination of Apr. 26, 2013, the patient had an OU NDS of 3 (FIG. 8B) and an OS VA of 20/80. Swelling and hemorrhages were reduced and arteriovenous crossings were normalizing. Following this examination the patient began taking Lipitor in addition to LZO3P.

Upon examination on May 28, 2013, the patient's OS VA was 20/40. However, the patient's OU NDS was 3.5 (FIG. 8C). Following treatment, the patient's choriodal swelling decreased and retinal swelling decreased (FIGS. 8D-F; choroid in upper right quadrant, retina in bottom left quadrant).

Upon examination Jul. 17, 2013, the patient's OS VA remained 20/40. However, the patient's OU NDS remained 3.5. It is contemplated that something interfered with nutrient absorption and/or nutrient delivery to the patient's eyes. Despite the continued NDS of 3.5, the patient's choriodal swelling and retinal swelling decreased further.

### Example 11

### LZO3P Treatment of Subjects Having Lutein and Zeaxanthin Deficiencies Can Prevent Retinal Detachment

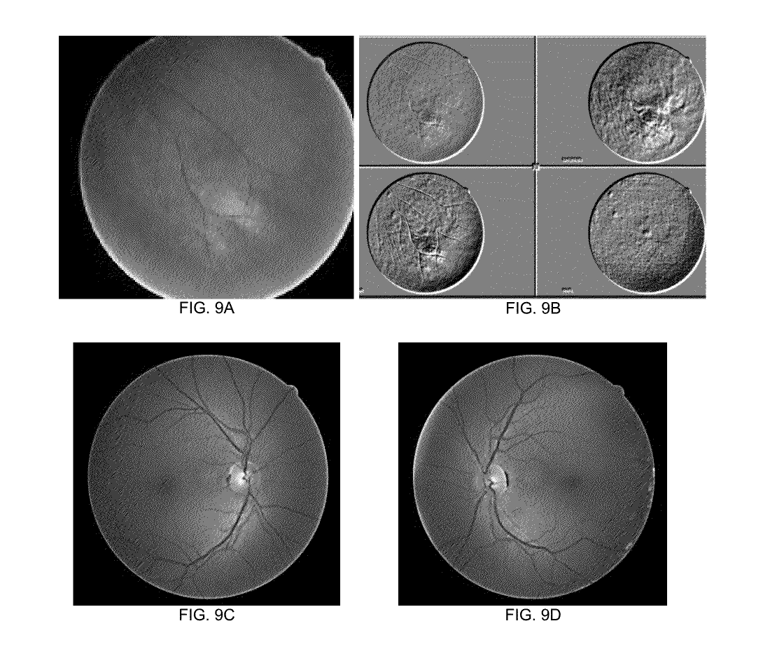

Subject Description: Patient 182 is a 63 year old male who was experiencing flashing lights in his vision. On May 15, 2013, examination revealed a horseshoe-shaped chorioretinal geographic atrophy in the inferior nasal peripheral retina (FIGS. 9A and B). The patient had an OD NDS of 4 (FIG. 9C) and an OS NDS of 3 (FIG. 9D). It is contemplated that the patient had OD MVNP, secondary to choroidal non-perfusion, partial retinal arterial arcade non-perfusion, or secondary to right carotid stenosis, (internal and/or common carotid) causing reduced perfusion on the right side. The patient's posterior pole was unremarkable.

Patient was prescribed LZO3P and was reexamined in August 2013. NDS of the peripheral retina improved from 4 to 3. Choroidal perfusion was improved. The smaller portion of the “horse shoe” seemed to be less atrophic. Posterior pole NDS became worse. It is contemplated that central retinal artery non-perfusion requires longer therapy of 6 to 12 months duration, to show improvement.

### Example 12

### Treatment of Subjects Having Lutein and Zeaxanthin Deficiencies Can Reverse Retinal Arteriole Scerlosis

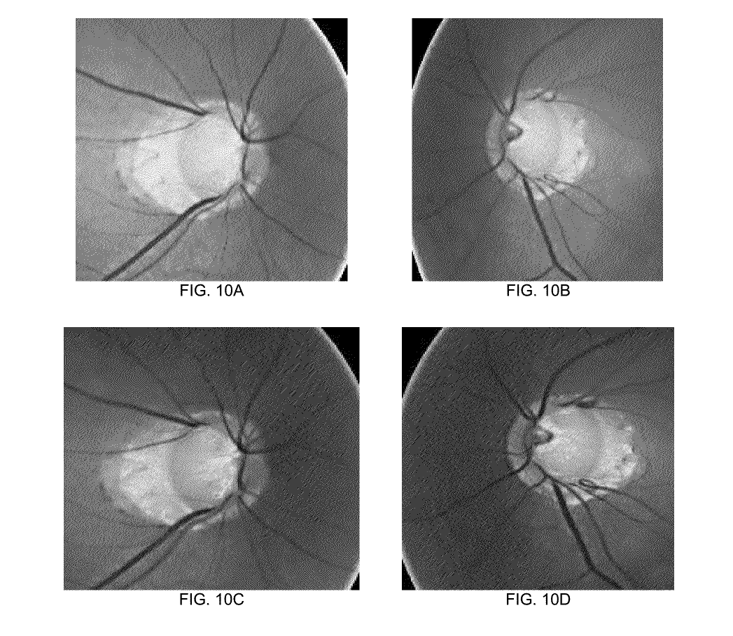

Subject Description: patient 531 is a 66 year old female. Upon initial examination on Oct. 4, 2012, the patient had an OU NDS of 5 (FIGS. 10A and B). Her central retinal arteries were one half occluded.

Treatment: LZO3P daily.

Results: Upon examination on May 2, 2013, the patient had an OU NDS of 2 (FIGS. 10C and D) and her arteries were fully perfused.

### Example 13

### LZO3P Treatment of Subjects Having Lutein and Zeaxanthin Deficiencies Can Reverse Presumed Choroidal Sclerosis

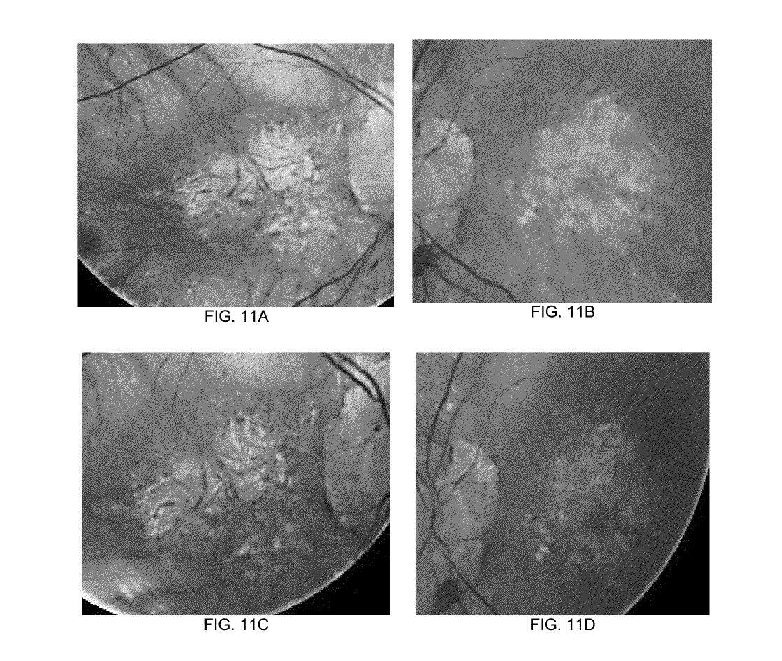

Subject Description: Patient 252 is an 86 year old female. Upon initial examination on Nov. 19, 2012, the patient had an OU NDS of 5 (FIGS. 11A and B). The patient exhibited non-perfusion, retina-RNFL fibrosis, excavation and VA LP OU.

Treatment: LZO3P daily.

Results: Upon examination on Mar. 18, 2013, the patient had an OU NDS of 3 (FIGS. 11C and D). The patient's choroidal blood vessels had increased in diameter, indicating reperfusion. Retina-RNFL fibrosis had reversed and excavation was reduced. No improvement of VA was apparent.

### Example 14

### Statin or LZO3P Treatment of Subjects Having Lutein and Zeaxanthin Deficiencies Can Reverse Presumed Unilateral Carotid Stenosis

Subject Description: Patient 0237 is a 63 year old female. Upon initial examination on Nov. 13, 2012, the patient had an OD NDS of 3 and an OS NDS of 5. The patient had a left retinal hemorrhage and was referred to a general practitioner for carotid assessment, wherein the patient was found to have a left carotid artery stenosis. The patient's general practitioner prescribed doubled dosage of the cholesterol medication that the patient was taking.

Upon examination on Feb. 19, 2013, the patient's OD NDS had improved to 2 and OS NDS had improved to 3. Further, the patient's left retinal hemorrhage had resolved. The statin medication the patient was taking appears to have resolved the presumed unilateral carotid stenosis and unilateral eye disease.

Patient 0895 is a 71 year old male. Upon initial examination on Sep. 27, 2010, the patient had mild OS ERM. OD NDS was 2, OS NDS was 4. Left partial carotid stenosis was presumed.

Treatment: Lutein (10 mg) and zeaxanthin (2 mg) daily.

Results: Upon examination on Oct. 4, 2011, the patient had an OD NDS of 1 and an OS NDS of 4. The mild OS ERM increased slightly. Twelve months on Lutein (10 mg) and zeaxanthin (2 mg) daily did not result in increased blood flow to the left eye.

Treatment: Lutein (20 mg) and zeaxanthin (5 mg) daily.

Upon examination on Apr. 16, 2012, the patient had an OD NDS of 2 and an OS NDS of 2. The patient's ocular nutrient health had improved and equalized and the patient's OS ERM had resolved. Lutein (20 mg) and zeaxanthin (5 mg) daily did result in increased blood flow to the left eye. It is contemplated that lutein (20 mg) and zeaxanthin (5 mg) daily can reverse partial carotid artery stenosis.

### Example 15

### Subject Who Declined Nutrient Therapy

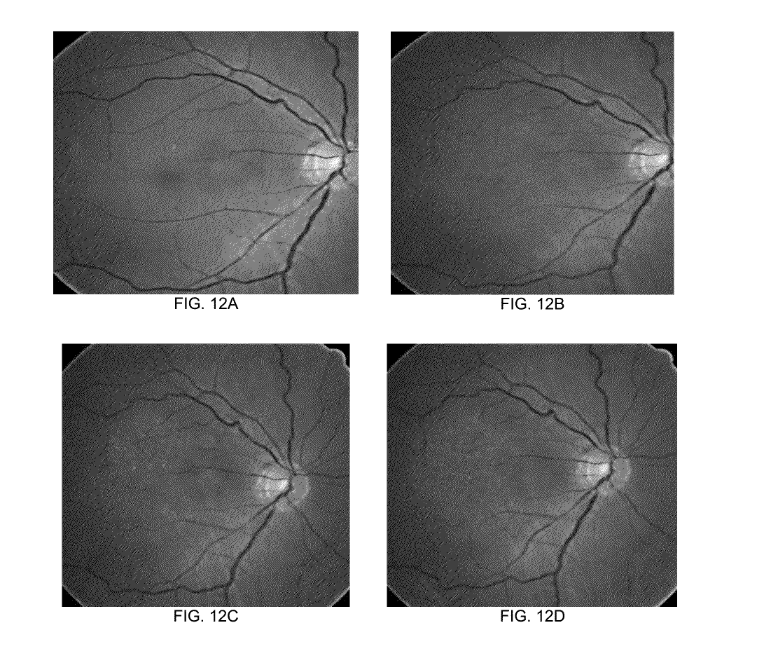

Subject Description: Patient 1423 is a 65 year old male. Upon initial examination on May 20, 2010, the patient exhibited an OU NDS 2, OU normal (FIG. 12A).

Upon examination on May 28, 2012, the patient had an OD NDS of 4 and an OS NDS of 3.5. The patient's OD had an ERM involving most of the posterior pole between the vascular arcades (FIG. 12B) and retinal puckering temporal to the optic nerve head. The patient declined nutrient therapy.

Upon examination on Nov. 29, 2012, the patient had an OD NDS of 4 and an OS NDS of 3.5. The patient's OD exhibited a large ERM with increasing fibrosis (FIG. 12C). The patient declined nutrient therapy.

Upon examination on May 30, 2013, the patient had an OD NDS of 4 and an OS NDS of 3. The patient's OD ERM remained large (FIG. 12D). The patient declined nutrient therapy.

### Example 16

### Perfusion Level of Various Vascular Supplies

It has been found that the pattern of light and dark in the retinal fibre layer can be analyzed in relation to the perfusion level in the macula, the superior central retinal artery, the inferior central retinal artery, the choriocapillaris (choroid) and the optic nerve head.

The results shown in table 3 demonstrate the perfusion level of various vascular supplies that occur in eyes at risk of eye disease and eyes with ocular diseases. These perfusion levels increase with nutrient supplementation as described elsewhere in this application. (Lower numeric values correspond with improved perfusion secondary to decreasing nutritional deficiency of lutein and zeaxanthin.)

In the table the patient's gender is indicated as 1 for female and 2 for male. The term “prevention” is used to indicate intervention when NDS of 4 or 5 is present in any vascular area. The term lens vacuole is used to indicate earliest sign of non-perfusion of interocular lens and occurs in eye with some scores of 4. In the cases described in table 3 the term VMT vitreomacular traction is epiretinal membranes.

TABLE 3

| | Reason for | | | | Right | | | | | Left | | | | |
|---|---|---|---|---|---|---|---|---|---|---|---|---|---|---|
| Subject | Intervention | F = 1 | Birth | Exam | Eye | sup | Inf | | | Eye | sup | Inf | | |
| No. | Prevention | M = 2 | Year | Date | Macula | CRA | CRA | Choroid | ONH | Macula | CRA | CRA | Choroid | ONH |
| 1 | | 2 | 1939 | Jan. 11, 2013 | 4 | 5 | 5 | 4 | 4 | 3 | 4 | 4 | 3 | 4 |
| | | | | Jan. 13, 2014 | 2 | 3 | 4 | 3 | 2 | 2 | 3 | 3 | 3 | 2 |
| 2 | | 1 | 1938 | Jan. 11, 2013 | 3 | 5 | 5 | 3 | 4 | 3 | 5 | 5 | 3 | 4 |
| | | | | Jan. 13, 2014 | 2 | 4 | 4 | 3 | 2 | 2 | 3 | 3 | 3 | 2 |
| 3 | | 1 | 1971 | May 23, 2013 | 4 | 3.5 | 3.5 | 4 | 3 | 4 | 3.5 | 3.5 | 4 | 3 |
| | | | | May 26, 2014 | 3 | 2 | 2 | 3 | 2 | 3 | 2 | 2 | 3 | 2 |
| 4 | lens | 1 | 1966 | May 15, 2013 | 5 | 3.5 | 3.5 | 5 | 3 | 3.2 | 4 | 4 | 4 | 3 |
| | vacuole | | | | | | | | | | | | | |
| | | | | Nov. 14, 2013 | 4 | 3.2 | 3.2 | 4 | 3 | 3 | 3.2 | 3.2 | 3.5 | 3 |
| 5 | left eye | 1 | 1966 | Apr. 11, 2014 | 2 | 3.5 | 3.5 | 3 | 2 | 2 | 4 | 4 | 4 | 2 |
| | VMT | | | | | | | | | | | | | |
| 6 | right eye | 2 | 1952 | May 8, 2013 | 3 | 3.2 | 3.7 | 4 | 3 | 3 | 3.7 | 3.5 | 4 | 3 |
| | | | | Nov. 22, 2013 | 3 | 3.5 | 3.5 | 3.5 | 3 | 3 | 3.5 | 3.5 | 3.5 | 3 |
| | | | | May 12, 2014 | 2 | 3 | 3 | 3 | 2 | 2 | 3 | 3 | 3 | 2 |
| 7 | left eye | 1 | 1946 | Nov. 26, 2013 | 4 | 4 | 5 | 4 | 3 | 4 | 4 | 5 | 4 | 3 |
| | | | | Jun. 24, 2014 | 3 | 3 | 3 | 3.5 | 2 | 3 | 3 | 4 | 3 | 2 |
| 8 | Dry AMD | 2 | 1944 | Mar. 25, 2013 | 5 | 3 | 3 | 4 | 4 | 5 | 3 | 3 | 4 | 4 |
| | drusen both | | | | | | | | | | | | | |
| | eyes | | | | | | | | | | | | | |
| | | | | Mar. 27, 2014 | 4 | 2 | 2 | 2 | 1 | 4 | 2 | 2 | 2 | 2 |
| 9 | Dry AMD | 1 | 1952 | Jun. 4, 2013 | 3 | 4 | 4 | 4 | 3 | 2 | 3.5 | 3.5 | 4 | 3 |
| | RPE left | | | | | | | | | | | | | |
| | eye | | | | | | | | | | | | | |
| | | | | Sep. 8, 2014 | 2 | 3 | 3.5 | 3 | 2 | 1 | 3.2 | 3.2 | 3 | 2 |
| 10 | Dry AMD | 2 | 1945 | May 23, 2013 | 4 | 4 | 4 | 5 | 4 | 4 | 3 | 3 | 3 | 3 |
| | GA right | | | | | | | | | | | | | |
| | eye | | | | | | | | | | | | | |
| | | | | Nov. 25, 2013 | 3 | 3 | 4 | 4 | 3 | 3 | 3 | 4 | 4 | 4 |
| | | | | May 26, 2014 | 3 | 3 | 4 | 3 | 3 | 3 | 2 | 3 | 2 | 2 |

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

We claim:

1. A method for identifying a subject having a nutritional deficiency, the method comprising:
a) obtaining an image of the subject's ocular posterior pole;
b) comparing the image to at least one reference ocular posterior pole image;
c) identifying a nutritional deficiency in the subject based on the comparison to the reference image,
wherein if the reference image is a healthy posterior pole, then an obtained image that comprises at least one region of increased contrast and/or decreased brightness relative to the reference image is indicative of the nutrient deficiency,
wherein if the reference image is a nutrient deficient posterior pole, then an obtained image that comprises at least one region of equal or increased contrast and/or equal or decreased brightness relative to the reference image is indicative of the nutrient deficiency, and
wherein the nutrient deficiency comprises a deficiency of one or more of lutein and zeaxanthin.

2. The method of claim 1, further comprising monitoring a subject for nutritional deficiency, wherein a subsequent image is obtained from the subject's posterior pole, compared with the image obtained in step a),
wherein if the subsequent image depicts increased contrast and/or decreased brightness relative to the image obtained in step a), then the subsequent image is indicative of lower nutrient levels in the subject's posterior pole, and
wherein if the subsequent image depicts decreased contrast and/or increased brightness relative to the image obtained in step a), then the subsequent image is indicative of higher nutrient levels in the subject's posterior pole.

3. The method of claim 1, further comprising assigning a score to the obtained image, wherein the score is based on a comparison between the obtained image and a reference database comprising posterior pole images obtained from a range of healthy and nutrient deficient subjects, wherein scores at opposite ends of the range are indicative of very healthy and severely nutrient deficient subjects, respectively.

4. The method of claim 3, wherein the assigned score is indicative of lutein and zeaxanthin levels in the subject's posterior pole.

5. The method of claim 3 wherein the assigned score is indicative of microvascular perfusion levels in the subject's posterior pole.

6. The method of claim 1 wherein in step a) the image is obtained is a color retinal photograph and wherein the photograph is converted to grayscale images of the choroid, the retina and the retinal nerve fiber layer (RNFL).

7. The method of claim 6 wherein in the RNFL grayscale image is compared to a reference RNFL grayscale image in step b).
